(19) United States
(12) Patent Application Publication
(10) Pub. No.: US 2025/0032796 A1
(43) Pub. Date: Jan. 30, 2025

(54) An Automated Process for Optimizing Spinal Cord Stimulation

(71) Applicant: The Regents of the University of California, Oakland, CA (US)

(72) Inventors: Prasad Shirvalkar, San Francisco, CA (US); Lawrence Poree, San Francisco, CA (US); Chad Sitgraves, San Francisco, CA (US)

(21) Appl. No.: 18/716,045

(22) PCT Filed: Dec. 6, 2022
(86) PCT No.: PCT/US2022/051957

§ 371 Date: Jun. 3, 2024

Related U.S. Application Data
(60) Provisional application No. 63/286,372, filed on Dec. 6, 2021.

(51) Int. Cl. A61N 1/36; A61N 1/05; A61N 1/372; G16H 10/60; G16H 20/40; G16H 40/63

(52) U.S. Cl. CPC A61N 1/36139; A61N 1/0551; A61N 1/36062; A61N 1/36071; A61N 1/36132; A61N 1/3615; A61N 1/36167; A61N 1/37247; G16H 10/60; G16H 20/40; G16H 40/63

(57) ABSTRACT

Methods and systems are provided for treating chronic pain using spinal cord stimulation in combination with a neural recording device that records electrical signals from neural activity associated with chronic pain. Machine learning computational models are used to classify patterns of neural activity associated with various pain states and pain relief. Neural signatures of “chronic pain” and “pain relief” are used to assist with spinal cord stimulation programming to determine therapeutic stimulation parameters that achieve analgesia without paresthesia, the minimum stimulation amplitude needed for effective therapy, and to predict whether spinal cord stimulation is likely to be effective in relieving pain. A closed-loop therapy system is provided comprising a spinal cord stimulator that records brain electrical signals from neural activity associated with pain and automatically adjusts spinal cord stimulator settings to deliver electrical stimulation to the spinal cord when pre-specified patterns of neural activity associated with pain are detected.

### CROSS-REFERENCE TO RELATED APPLICATION

This application claims benefit under 35 U.S.C. § 119(e) of provisional application 63/286,372, filed Dec. 6, 2021, which application is hereby incorporated by reference in its entirety.

### INTRODUCTION

Chronic pain affects more people than heart disease, diabetes and cancer combined and will affect 1 in 4 Americans over their lifetime (CDC (2016) Wide-ranging online data for epidemiologic research (WONDER), 2016). Treating pain with chronic opioid prescriptions has led to the current opioid epidemic, which is now the leading killer of Americans under age 50. Clearly, the need exists for better pain treatments that are personalized for each patient. In the last 20 years, a revolutionary opioid-sparing therapy for many chronic pain conditions including lower back pain (13.1% US prevalence, Shmagel et al. (2016) Arthritis Care Res. 68:1688-1694) involves permanent electrical stimulation of the spinal cord (Kumar et al. (2007) Pain 132:179-188). Presently, spinal cord stimulation (SCS) uses tonic or intermittent waveforms of electrical stimulation that differentially produce a paresthesia sensation, however the neurophysiological basis of how SCS produces pain relief and the relationship of paresthesia perception to analgesia are still poorly understood. SCS therapy is widely believed to impart analgesia through the production of pain-masking sensory paresthesia in the body region of interest; however, it is possible to deliver therapeutic stimulation that significantly reduces pain even in the absence of perceptible sensory changes (Wolter et al. (2012) Eur. J. Pain 16:648-655; Mekhail et al. (2020) Neuromodulation 23(2):185-195) Although such ‘paresthesia-free’ stimulation often relies on high frequency stimulation or alternative waveforms, paresthesia-free analgesia can also be achieved using traditional tonic waveforms simply with lower amplitudes. Lowering the amplitude of stimulation can also increase battery life, reduce side effects of stimulation, and potentially avert the onset of tolerance over time by reducing electrical dosage and nervous system adaptation.

Of the patients that receive permanent SCS implants, over 70% report a reduction in pain of greater than 50%. However, SCS therapy trials following the current standard of care have an industry-wide failure rate as high as 52% (Deer et al. (2014) Neuromodulation J. Int. Neuromodulation Soc. 17:515-50). Further, of the patients that receive permanent SCS implants, most experience a reduction in efficacy greater than 50% within 2 years (Wolter et al. (2012) Eur. J. Pain 16:648-655, Mekhail et al. (2020) Neuromodulation 23(2):185-195), potentially due to nervous system adaptation to electrical stimulation over time.

Thus, there remains a need for better methods of delivering SCS therapy and relieving chronic pain.

### SUMMARY

Methods and systems are provided for treating chronic pain using spinal cord stimulation. In particular, spinal cord stimulation is performed with a neural recording device that records electrical signals from neural activity associated with chronic pain. Machine learning computational models are used to detect and classify patterns of neural activity associated with various pain states and pain relief. The neural signatures of “chronic pain” and “pain relief” are used to assist with spinal cord stimulation programming to determine therapeutic stimulation parameters that achieve analgesia without paresthesia and the minimum stimulation amplitude needed for effective therapy. The methods and systems can be used in performing open-loop therapy to provide clinical guidance to clinicians or technicians for adjusting spinal cord stimulation programming. Methods and systems are also provided for performing closed-loop therapy with a spinal cord stimulator that records brain electrical signals from neural activity associated with pain and automatically adjusts spinal cord stimulator settings and/or delivers electrical stimulation to the spinal cord when pre-specified patterns of neural activity associated with pain are detected. Such closed-loop methods may also include automated provision of clinical guidance for stimulation programming or direct computer-to-computer implementation of programming.

Methods are also disclosed for mapping the brain of a patient to optimize placement of electrodes for detecting neural activity associated with pain and/or relief of pain. Placement of electrodes for delivery of electrical stimulation to the spinal cord may also be adjusted to optimize relief of pain from electrical stimulation. Methods of monitoring and evaluating the effects of electrical stimulation on sensory perception of pain are also provided.

The methods and systems can be used for treatment of pain, including pain caused by any pain-associated disorder such as, but not limited to, back pain, failed back surgery syndrome, spinal cord injury, spinal stenosis, post-surgical pain, complex regional pain syndrome, arachnoiditis, angina, nerve-related pain (e.g., such as caused by diabetic neuropathy, cancer-related neuropathy, or nerve damage caused by radiation, surgery, or chemotherapy), peripheral vascular disease, pain after an amputation, visceral abdominal pain, perineal pain, multiple sclerosis, arthritis, or chronic leg (e.g., sciatica) or arm pain.

In one aspect, a method for treating chronic pain in a subject is provided, the method comprising: positioning a first electrode at a location in the epidural space to deliver electrical stimulation to the spinal cord of the subject; positioning a second electrode at a location in a frontal lobe region of the brain of the subject to detect a brain electrical signal associated with the chronic pain; detecting the brain electrical signal at the frontal lobe region of the brain of the subject using the second electrode; and applying electrical stimulation to the spinal cord using the first electrode in a manner effective to treat the chronic pain in the subject when the brain electrical signal, detected using the second electrode, exceeds a threshold level.

In certain embodiments, the method further comprises using a control algorithm to automate said applying electrical stimulation when the brain electrical signal exceeds a threshold level.

In certain embodiments, the control algorithm uses a machine learning algorithm for pain classification. In some embodiments, the machine learning algorithm is a supervised machine learning algorithm. In some embodiments, the supervised machine learning algorithm is a support vector machine (SVM) learning algorithm.

In certain embodiments, the control algorithm further modulates one or more programmed stimulation parameters based on a level of power of the brain electrical signal.

In certain embodiments the algorithm provides updated optimal stimulation setting recommendations to the clinician for guiding programing and decision making.

In certain embodiments, the method further comprises determining the minimum stimulation amplitude needed to relieve the chronic pain based on the level of power of the brain electrical signal.

In certain embodiments, the electrical stimulation is applied to the spinal cord at the minimum stimulation amplitude needed to relieve the chronic pain.

In certain embodiments, the method further comprises positioning a plurality of electrodes at the location in the frontal lobe region of the brain of the subject for detection of the brain electrical signal by stereoelectroencephalography (sEEG).

In certain embodiments, the frontal lobe region is a right frontal lobe region of the brain.

In certain embodiments, the electrical stimulation is applied unilaterally or bilaterally.

In certain embodiments, the brain electrical signal comprises alpha frequency, beta frequency, gamma frequency, delta frequency, or theta frequency neural oscillations. In some embodiments, the theta frequency neural oscillations are in a range from 4 Hz to 8 Hz. In other embodiments, the beta frequency neural oscillations are in a range from 12 Hz to 30 Hz.

In certain embodiments, the second electrode is placed on a surface of the frontal lobe region or within the frontal lobe region.

In certain embodiments, the second electrode is a non-brain penetrating surface electrode array or a brain-penetrating electrode array.

In certain embodiments, the method further comprises positioning a third electrode at a location in a left frontal cortex region of a brain of the subject to detect a brain electrical signal associated with relief of the chronic pain. In some embodiments, the third electrode is placed on a surface of the left frontal cortex region or within the left frontal cortex region. In some embodiments, the third electrode is a non-brain penetrating surface electrode array or a brain-penetrating electrode array.

In certain embodiments, the method further comprises using a control algorithm to automate adjustment of one or more programmed stimulation parameters to maintain the level of the brain electrical signal associated with relief of the chronic pain in a target range.

In certain embodiments, the method further comprises determining a paresthesia threshold for the electrical stimulation; and using a control algorithm to automate adjustment of one or more programmed stimulation parameters to apply the electrical stimulation at a level below the paresthesia threshold.

In certain embodiments, the chronic pain is caused by a pain-associated disorder, wherein applying the electrical stimulation relieves the pain. In some embodiments, the pain-associated disorder is back pain, failed back surgery syndrome, spinal cord injury, spinal stenosis, post-surgical pain, complex regional pain syndrome, arachnoiditis, angina, nerve-related pain (e.g., such as caused by diabetic neuropathy, cancer-related neuropathy, or nerve damage caused by radiation, surgery, or chemotherapy), peripheral vascular disease, pain after an amputation, visceral abdominal pain, perineal pain, multiple sclerosis, arthritis, or chronic leg (e.g., sciatica) or arm pain.

In certain embodiments, the method further comprises assessing effectiveness of the treatment in the subject.

In certain embodiments, the method further comprises mapping the brain of the subject to identify an optimal location in the right frontal lobe region to detect the brain electrical signal associated with the chronic pain.

In certain embodiments, the method further comprises mapping the brain of the subject to identify an optimal location in the left frontal cortex region to detect the brain electrical signal associated with relief of the chronic pain.

In certain embodiments, the method further comprises assessing relief of pain during or after treatment of the subject by using a visual analog scale or a verbal rating scale.

In certain embodiments, the method further comprises repositioning the first electrode in the epidural space to improve relief of pain.

In certain embodiments, the first electrode is placed at a location in the epidural space at a lower thoracic level, a mid-thoracic level, an upper thoracic level, or a cervical level of the spine.

In certain embodiments, a method of detecting whether a subject who has chronic pain is responding to spinal cord stimulation therapy is provided, the method comprising: positioning a first electrode at a location in the epidural space to deliver electrical stimulation to the spinal cord of the subject; positioning a second electrode at a location in a frontal lobe region of the brain of the subject to detect a brain electrical signal associated with the chronic pain; detecting the brain electrical signal in the frontal lobe region of the brain of the subject using the second electrode before and after applying electrical stimulation to the spinal cord using the first electrode, wherein a decrease in level of power of the brain electrical signal indicates the subject is responding to the spinal cord stimulation therapy and an increase or no change in the level of power of the brain electrical signal indicates the subject is not responding to the spinal cord stimulation therapy.

In another aspect, a system for treating chronic pain in a subject is provided, the system comprising: a first electrode adapted for positioning at a location in the epidural space to deliver electrical stimulation to the spinal cord of a subject; a second electrode adapted for positioning at a frontal lobe region of the brain of the subject and for detecting a brain electrical signal from the frontal lobe region of the brain of the subject; and a processor programmed to instruct the first electrode to apply an electrical stimulation to the spinal cord in a manner effective to treat the chronic pain in the subject when a brain electrical signal that exceeds a threshold level is detected using the second electrode.

In certain embodiments, the frontal lobe region is a right frontal lobe region of the brain.

In certain embodiments, the brain electrical signal comprises alpha frequency, beta frequency, gamma frequency, delta frequency, or theta frequency neural oscillations.

In certain embodiments, the second electrode is a non-brain penetrating surface electrode array or a brain-penetrating electrode array.

In certain embodiments, the system further comprises a third electrode adapted for positioning at a location in a left frontal cortex region of the brain of the subject. In some embodiments, the third electrode is a non-brain penetrating surface electrode array or a brain-penetrating electrode array.

In certain embodiments, the system further comprises a user interface comprising an input electronically coupled to the processor for instructing the first electrode to apply an electrical stimulation to the spinal cord to treat the chronic pain in the subject. In some embodiments, the user interface is password protected and is operable by a health care practitioner.

In certain embodiments, the processor is further programmed to modulate one or more programmed stimulation parameters according to an algorithm control law; and apply the modulated electrical stimulation to the spinal cord using the first electrode in a manner effective to treat the chronic pain.

In certain embodiments, the processor is further programmed to set a maximum number of electrical stimulations per day.

In certain embodiments, the processor is further programmed to set a total amount of time of electrical stimulation per day.

In another aspect, a computer implemented method for programming a spinal cord stimulator (SCS) to relieve chronic pain in a subject is provided, the computer performing steps comprising: receiving recorded brain electrical signal data from a frontal lobe region of the brain of the subject; analyzing the recorded brain electrical signal data using a pain classification model that identifies patterns of electrical signals in the recorded brain electrical signal data associated with chronic pain; adjusting one or more programmed stimulation parameters based on the recorded brain electrical signal data according to an algorithm control law; and instructing the spinal cord stimulator to apply an electrical stimulation to the spinal cord to treat the chronic pain in the subject.

In certain embodiments, a machine learning algorithm is used to generate the pain classification model. In some embodiments, the machine learning algorithm is a support vector machine (SVM) learning algorithm.

In certain embodiments, the computer implemented method further comprises receiving recorded brain electrical signal data from a frontal cortex region of the brain of the subject; and adjusting of one or more programmed stimulation parameters to maintain a level of a brain electrical signal associated with relief of the chronic pain in a target range.

In certain embodiments, the computer implemented method further comprises determining a minimum stimulation amplitude needed to relieve the chronic pain based on the recorded brain electrical signal data; and instructing the spinal cord stimulator to apply an electrical stimulation to the spinal cord at the minimum stimulation amplitude needed to relieve the chronic pain.

In certain embodiments, the computer implemented method further comprises receiving inputted data comprising a paresthesia threshold for the electrical stimulation; and instructing the spinal cord stimulator to apply the electrical stimulation at a level below the paresthesia threshold.

In certain embodiments, the computer implemented method further comprises processing the recorded brain electrical signal data to remove non-physiological frequencies from 1 Hz to 500 Hz.

In certain embodiments, the computer implemented method further comprises performing baseline correction on the recorded brain electrical signal data.

In certain embodiments, the computer implemented method further comprises removing ambient electrical line noise from the recorded brain electrical signal data. In some embodiments, ambient electrical line noise is removed using a Notch filter at 60 Hz and harmonics.

In certain embodiments, the computer implemented method further comprises removing a first minute and a last minute from the recorded brain electrical signal data.

In certain embodiments, the computer implemented method further comprises performing independent component and time-locked coherence analyses to detect and reduce artifacts caused by blink and pulse activity.

In certain embodiments, the computer implemented method further comprises splitting the recorded brain electrical signal data into consecutive time epochs. In some embodiments, each time epoch comprises 1 second of time of the recorded brain electrical signal data. In some embodiments, the computer implemented further comprises performing frequency decomposition to calculate average power in each neurophysiological activity frequency band for each time epoch. In some embodiments, the computer implemented method further comprises: calculating spectral tilt values for each epoch; and fitting the spectral tilt values to the pain classification model to determine how to adjust one or more programmed stimulation parameters.

In certain embodiments, the computer implemented method further comprises: a) ranking predicted stimulation effectiveness for available settings of the spinal cord stimulator based on classifier scores for stimulation effectiveness of each setting using a linear classification model; b) selecting stimulation settings predicted to have highest stimulation effectiveness based on the linear classification model; c) receiving recorded brain electrical signal data from the frontal lobe region of the brain of the subject after applying electrical stimulation with the spinal cord stimulator to the spinal cord using the settings predicted to have highest stimulation effectiveness; d) analyzing the recorded brain electrical signal data to evaluate neural response of the subject to the electrical stimulation; e) updating the linear classification model based on the neural response of the subject to the electrical stimulation to generate an updated linear classification model; f) updating the ranking of predicted stimulation effectiveness for the available settings of the spinal cord stimulator using the updated linear classification model; g) selecting stimulation settings predicted to have the highest stimulation effectiveness based on the updated linear classification model; h) receiving recorded brain electrical signal data from the frontal lobe region of the brain of the subject after applying the electrical stimulation with the spinal cord stimulator to the spinal cord using the settings predicted to have highest stimulation effectiveness based on the updated linear classification model; and i) repeating e)-h) to adjust the available settings of the spinal cord stimulator to optimize stimulation effectiveness.

In certain embodiments, the computer implemented method further comprises: predicting whether a subject who has chronic pain will respond to the spinal cord stimulation based on the classifier scores for stimulation effectiveness.

In certain embodiments, the computer implemented method further comprises storing a user profile for the subject comprising information regarding the recorded brain electrical signal data associated with the chronic pain.

In certain embodiments, the computer implemented method further comprises storing a user profile for the subject comprising information regarding the programmed stimulation parameters used to apply electrical stimulation to the spinal cord to treat the chronic pain in the subject based on the recorded brain electrical signal data.

In another aspect, a non-transitory computer-readable medium is provided, the non-transitory computer-readable medium comprising program instructions that, when executed by a processor in a computer, causes the processor to perform a computer implemented method described herein.

In another aspect, a kit comprising the non-transitory computer-readable medium described herein and instructions for treating chronic pain in a subject with a spinal cord stimulator is provided.

In another aspect, a system for treating chronic pain in a subject is provided, the system comprising: a spinal cord stimulator comprising a first electrode adapted for positioning at a location in the epidural space to deliver electrical stimulation to the spinal cord of a subject; a neural recording device comprising a second electrode adapted for positioning at a frontal lobe region of the brain of the subject for recording brain electrical signal data from the frontal lobe region of the brain of the subject; and a processor programmed according to a computer implemented method described herein to adjust one or more stimulation parameters based on the recorded brain electrical signal data and instruct the first electrode to apply an electrical stimulation to the spinal cord.

In another aspect, a kit is provided comprising a system, described herein, for treating chronic pain in a subject and instructions for treating chronic pain in a subject with a spinal cord stimulator.

### BRIEF DESCRIPTION OF THE DRAWINGS

FIG. 1. Example Independent Components ranked from highest to lowest weight for source reconstruction analysis.

FIGS. 2A-2E. Example Neural Signature of Pain Relief with SCS. (FIG. 2A) Left-X-ray of Spine with implanted SCS electrodes, Right-Patient drawing of leg pain area shaded black. (FIG. 2B-2E) Overhead scalp heatmaps of averaged EEG activity. Baseline band-limited neural activity is strongest over right frontal lobe (FIG. 2B, red=higher power), when pain is rated 7/10 similar to ineffective (FIG. 2C) or subthreshold SCS (FIG. 2D). With optimal SCS parameters that reduce pain to 4/10, (FIG. 2E) band-limited activity is highest over left frontal cortex, providing a key biomarker for this subject.

FIG. 3A. Confusion Matrix of Classification Accuracy for various stimulation amplitudes. (B is baseline, A1-6 indicate amplitudes; A6 is perception threshold) Bottom Panel shows ROC curves. FIG. 3B. Example of spectral tilt measurements made on each contact of an EEG cap. Tilt/Slope values are indicated overlying each plot.

FIGS. 4A-4C. Automated SCS Logic Diagrams. (FIG. 4A) A workflow diagram showing a comparison of current practice and automated SCS, including methods of predicting trial success. (FIG. 4B) A workflow diagram showing a comparison of current practice and automated SCS, including methods of adjusting stimulation parameters. (FIG. 4C) A workflow diagram showing fully automated SCS using EEG data to adjust SCS settings.

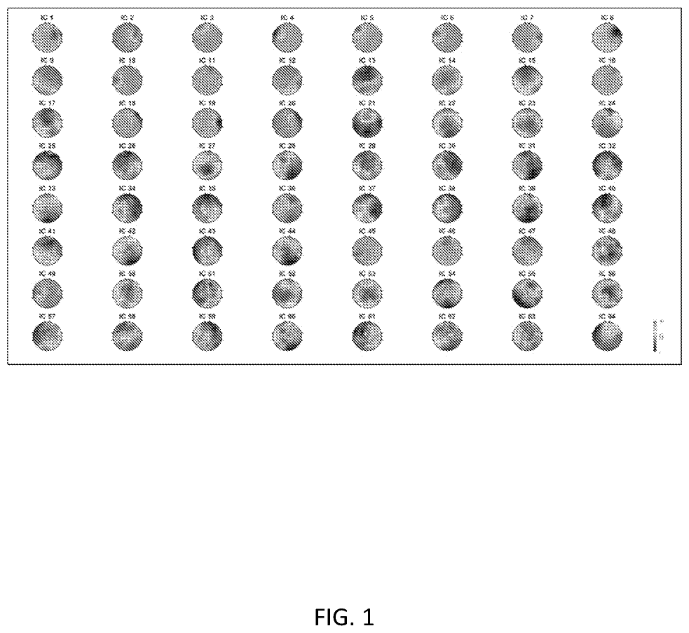

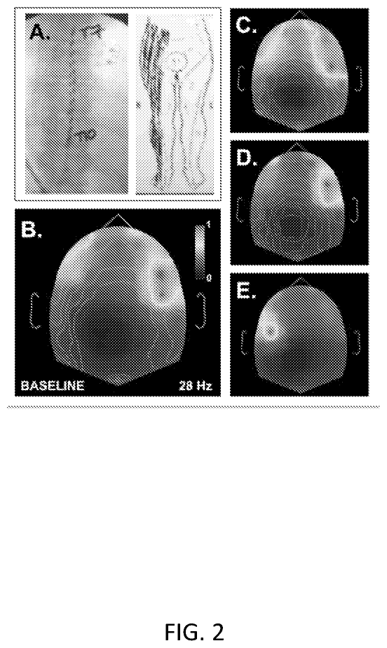

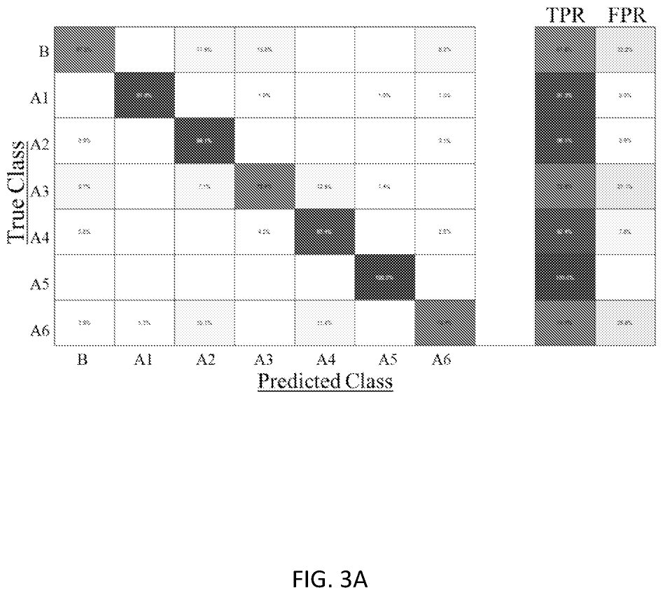

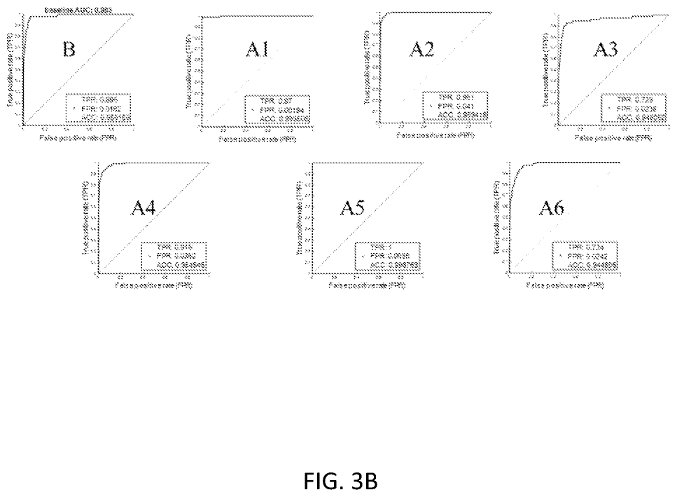

### DETAILED DESCRIPTION

Methods and systems are provided for treating chronic pain using spinal cord stimulation. In particular, spinal cord stimulation is performed with a neural recording device that records electrical signals from neural activity associated with chronic pain. Machine learning computational models are used to detect and classify patterns of neural activity associated with various pain states and pain relief. The neural signatures of “chronic pain” and “pain relief” are used to assist with spinal cord stimulation programming to determine therapeutic stimulation parameters that achieve analgesia without paresthesia and the minimum stimulation amplitude needed for effective therapy. The methods and systems can be used in performing open-loop therapy to provide clinical guidance to clinicians or technicians for adjusting spinal cord stimulation programming. Methods and systems are also provided for performing closed-loop therapy with a spinal cord stimulator that records brain electrical signals from neural activity associated with pain and automatically adjusts spinal cord stimulator settings and/or delivers electrical stimulation to the spinal cord when pre-specified patterns of neural activity associated with pain are detected. Such closed-loop methods may also include automated provision of clinical guidance for stimulation programming or direct computer-to-computer implementation of programming.

Pain-associated disorders that can be treated with the systems and methods disclosed herein include, without limitation, back pain, failed back surgery syndrome, spinal cord injury, spinal stenosis, post-surgical pain, complex regional pain syndrome, arachnoiditis, angina, nerve-related pain (e.g., such as caused by diabetic neuropathy, cancer-related neuropathy, or nerve damage caused by radiation, surgery, or chemotherapy), peripheral vascular disease, pain after an amputation, visceral abdominal pain, perineal pain, multiple sclerosis, arthritis, neck pain, or chronic leg (e.g., sciatica) or arm pain.

Before exemplary embodiments of the present invention are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “an electrode” or “the electrode” includes a plurality of such electrodes and reference to “an electrical signal” or “the electrical signal” includes reference to one or more electrical signals, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. To the extent such publications may set out definitions of a term that conflicts with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

The term “pain-associated disorder” is used herein to refer to conditions that cause pain in a person suffering from the disorder. The pain may be chronic, idiopathic, nociceptive, inflammatory, visceral or neuropathic pain. Pain-associated disorders include, without limitation, back pain, failed back surgery syndrome, spinal cord injury, spinal stenosis, post-surgical pain, complex regional pain syndrome, arachnoiditis, angina, nerve-related pain (e.g., such as caused by diabetic neuropathy, cancer-related neuropathy, or nerve damage caused by radiation, surgery, or chemotherapy), peripheral vascular disease, pain after an amputation, visceral abdominal pain, perineal pain, multiple sclerosis, arthritis, or chronic leg pain (e.g., sciatica), neck pain, foot pain, or arm pain.

By “treatment” or “treating” is meant that at least an amelioration of pain associated with the condition afflicting the subject is achieved such that the patient has a desired or beneficial clinical result, where amelioration refers to at least a reduction in the magnitude of a parameter, e.g., pain associated with the condition being treated. As such, treatment includes a broad spectrum of situations ranging from lessening intensity, duration or extent of pain caused by a condition and/or correlated with a condition, up to and including completely eliminating the pain, along with any associated symptoms. Treatment therefore includes situations where the condition, or at least pain associated therewith, is completely inhibited, e.g., prevented from happening, or stopped, e.g., terminated, such that the subject no longer suffers from the condition, or at least the pain symptoms that characterize the condition. Treatment also includes situations where the progression of the condition, or at least the progression of a symptom associated therewith, is slowed, delayed, or halted. In such cases, a subject might still have residual symptoms associated with the pathological condition, but any increase in the severity or magnitude of the symptoms is slowed, delayed, or prevented.

The term “subject” as used herein refers to a patient in need of the treatments disclosed herein. The patient may be a mammal, such as, a rodent, a feline, a canine, a primate, or a human, e.g., a child, an adolescent, an adult, such as a young, middle-aged, or elderly human. The patient may have been diagnosed as having pain or may be suspected of suffering from a pain-associated disorder.

The term “user” as used herein refers to a person that interacts with a device and/system disclosed herein for performing one or more steps of the presently disclosed methods. The user may be the patient receiving treatment. The user may be a health care practitioner, such as, the patient's physician.

### Methods

The present disclosure provides methods for ameliorating pain using spinal cord stimulation. In particular, spinal cord stimulation is performed with a neural recording device that records electrical signals from neural activity associated with chronic pain. Machine learning computational models are used to detect and classify patterns of neural activity associated with various pain states and pain relief. The neural signatures of “chronic pain” and “pain relief” are used to assist with spinal cord stimulation programming to determine therapeutic stimulation parameters that achieve analgesia without paresthesia and the minimum stimulation amplitude needed for effective therapy. The methods and systems can be used in performing open-loop therapy to provide clinical guidance to clinicians or technicians for adjusting spinal cord stimulation programming. Methods and systems are also provided for performing closed-loop therapy with a spinal cord stimulator that records brain electrical signals from neural activity associated with pain and automatically adjusts spinal cord stimulator settings and/or delivers electrical stimulation to the spinal cord when pre-specified patterns of neural activity associated with pain are detected. Such closed-loop methods may also include automated provision of clinical guidance for stimulation programming or direct computer-to-computer implementation of programming.

The subject methods can be used to treat any condition causing pain, including chronic, idiopathic, nociceptive, or neuropathic pain. Pain-associated disorders which may be treated with spinal cord stimulation, as described herein, include, without limitation, back pain, failed back surgery syndrome, spinal cord injury, spinal stenosis, post-surgical pain, complex regional pain syndrome, arachnoiditis, angina, nerve-related pain (e.g., such as caused by diabetic neuropathy, cancer-related neuropathy, or nerve damage caused by radiation, surgery, or chemotherapy), peripheral vascular disease, pain after an amputation, visceral abdominal pain, perineal pain, multiple sclerosis, arthritis, or chronic leg pain (e.g., sciatica), neck pain, foot pain, or arm pain. Various steps and aspects of the methods will now be described in greater detail below.

The method includes positioning a first electrode in the epidural space to deliver electrical stimulation to the spinal cord (i.e., SCS electrode) and positioning a second electrode at the frontal lobe region of the brain of the subject to detect brain electrical signals from neural activity associated with pain (i.e., detection electrode). In some embodiments, one or more SCS electrodes are positioned in the epidural space, and one or more detection electrodes are positioned at the frontal lobe region of the brain of the subject. The detection electrodes may be non-brain penetrating surface electrodes or brain-penetrating depth electrodes. The electrical stimulation may be applied to the spinal cord using the SCS electrode in a manner effective for ameliorating pain in a subject having a pain-associated disorder when a brain electrical signal is detected from the frontal lobe region of the brain using the detection electrode that has a signal amplitude exceeding a threshold level indicating that the subject has a level of pain severity in need of treatment.

In certain embodiments, one or more detection electrodes are used to record brain electrical signals for neural activity associated with pain or pain relief in one or more brain regions. A detection electrode may be placed, for example, in the right frontal lobe region to detect neural activity associated with pain, and/or in the left frontal cortex region to detect neural activity associated with relief of pain, or in other regions of the brain suitable for detection. The site chosen for detection may differ for different subjects and may depend on mapping of the brain of an individual subject to identify the optimal location for positioning an electrode for detecting brain electrical signals from neural activity associated with pain or pain relief, as discussed further below.

As used herein, the phrases “an electrode” or “the electrode” refer to a single electrode or multiple electrodes such as an electrode array. As used herein, the term “contact” as used in the context of an electrode in contact with a region of the brain or epidural space refers to a physical association between the electrode and the region. In other words, a detection electrode that is in contact with a region of the brain is physically touching the region of the brain. An SCS electrode in the epidural space can conduct electricity into the spinal cord. Electrodes used in the methods disclosed herein may be monopolar (cathode or anode) or bipolar (e.g., having an anode and a cathode).

Positioning a detection electrode for recording neural activity at specified region(s) of the brain may be carried out using standard surgical procedures for placement of intra-cranial electrodes. In certain cases, placing the detection electrode may involve positioning the electrode on the surface of the specified region(s) of the brain. For example, electrodes may be placed on the surface of the brain at the right frontal lobe, the left frontal cortex, or any combination thereof. The electrode may contact at least a portion of the surface of the brain at the right frontal lobe or the left frontal cortex. In some embodiments, the electrode may contact substantially the entire surface area at the right frontal lobe or the left frontal cortex. In some embodiments, the electrode may additionally contact area(s) adjacent to the right frontal lobe or the left frontal cortex regions. In some embodiments, an electrode array arranged on a planar support substrate may be used for detecting brain electrical signals for neural activity from one or more of the brain regions specified herein. The surface area of the electrode array may be determined by the desired area of contact between the electrode array and the brain. An electrode for implanting on a brain surface, such as, a surface electrode or a surface electrode array may be obtained from a commercial supplier. A commercially obtained electrode/electrode array may be modified to achieve a desired contact area. In some cases, the non-brain penetrating electrode (also referred to as a surface electrode) that may be used in the methods disclosed herein may be an electrocorticography (ECoG) electrode or an electroencephalography (EEG) electrode. In certain embodiments, a plurality of electrodes is positioned at one or more of the brain regions specified herein for detection of electroencephalographic signals by stereoelectroencephalography (sEEG).

In certain cases, placing the detection electrode at a target area or site (e.g., the right frontal lobe or the left frontal cortex) may involve positioning a brain penetrating electrode (also referred to as depth electrode) in the specified region(s) of the brain. For example, a detection electrode may be placed in the right frontal lobe or the left frontal cortex region. In some embodiments, the detection electrode may additionally contact area(s) adjacent to the right frontal lobe or the left frontal cortex. In some embodiments, an electrode array may be used for detecting neural activity from the right frontal lobe or the left frontal cortex, or a combination thereof, as specified herein.

The depth to which a detection electrode is inserted into the brain may be determined by the desired level of contact between the electrode array and the brain. A brain-penetrating electrode array may be obtained from a commercial supplier. A commercially obtained electrode array may be modified to achieve a desired depth of insertion into the brain tissue.

Positioning an electrode in the epidural space for delivering electrical stimulation to the spinal cord may be carried out using standard surgical procedures for placement of electrodes for spinal cord stimulation. The placement of the SCS electrodes depends on the location where the subject is experiencing pain. SCS electrodes should be placed to allow stimulation of nerves in the area where pain is felt. SCS electrodes are placed at the appropriate level along the spine to relieve pain. For example, the electrode may be placed in the epidural space between vertebrae T9 to T10 to relieve lower back pain, at lower thoracic levels for lower limb pain, and at cervical levels for upper limb pain. Paddle electrodes or percutaneous electrodes may be used for spinal cord stimulation. Fluoroscopy or ultrasound may be used to provide guidance for placement of the SCS electrodes. In addition, a pulse generator is placed under the skin, typically near the buttocks or abdomen. The surgical procedure may involve an incision over the spine for placement of the SCS electrodes. An electrode lead is tunneled under the skin and connected to the pulse generator. A subcutaneous pocket can be formed in the abdominal-flank area or the upper buttock for implantation of the pulse generator.

Current is supplied by the pulse generator to the SCS electrodes. Parameters such as pulse width, frequency, and amplitude can be adjusted in response to changes in neural activity in the right frontal lobe and/or the left frontal cortex to optimize pain relief. In some embodiments, a closed loop system is used to adjust SCS settings automatically in response to changes in neural activity in the right frontal lobe and/or the left frontal cortex. In other embodiments, an open loop system is used in which SCS settings are adjusted by a user or medical practitioner based on the neural activity in the right frontal lobe and/or the left frontal cortex.

The electrical stimulation may be applied using a single electrode, electrode pairs, or an electrode array. In some embodiments, the number of electrodes used to deliver electrical stimulation to the spinal cord ranges from 8 to 32, including any number of electrodes in this range such as 8, 10, 12, 14, 16, 18, 20, 22, 24, 26, 28, 30, or 32 electrodes. In some embodiments, the electrical stimulation is applied to more than one site along the spinal cord. The site to which the electrical stimulation is applied may be alternated or otherwise spatially or temporally patterned. Electrical stimulation may be applied to the sites simultaneously or sequentially. In certain embodiments, the region of the spine to which electrical stimulation is applied is at lower thoracic levels, mid-thoracic levels, upper thoracic levels, cervical levels, or other regions of the spine suitable for stimulation. The site chosen for stimulation may differ for different subjects and will depend on mapping of the epidural space of an individual subject to identify the optimal location for positioning an electrode for delivery of electrical stimulation to ameliorate pain.

In some embodiments, an electrode array arranged on a planar support substrate may be used for electrically stimulating the spinal cord. The surface area of the electrode array may be determined by the desired area of contact between the electrode array and the spine. In some cases, cylindrical electrode arrays, paddle-style electrode arrays, or plate-style electrode arrays may be used in the methods disclosed herein for spinal cord stimulation. Such SCS electrode arrays for implanting in the epidural space, may be obtained from a commercial supplier. A commercially obtained electrode/electrode array may be modified to achieve a desired contact area.

The precise number of SCS electrodes or detection electrodes contained in an electrode array (e.g., for electrical stimulation or detection of neural activity) may vary. In certain aspects, an electrode array may include two or more electrodes, such as 3 or more, including 4 or more, e.g., about 3 to 6 electrodes, about 6 to 12 electrodes, about 12 to 18 electrodes, about 18 to 24 electrodes, about 24 to 30 electrodes, about 30 to 48 electrodes, about 48 to 72 electrodes, about 72 to 96 electrodes, or about 96 or more electrodes. The electrodes may be arranged into a regular repeating pattern (e.g., a grid, such as a grid with about 1 cm spacing between electrodes), or no pattern. An electrode that conforms to the target site for optimal delivery of electrical stimulation may be used. One such example, is a single multi contact electrode with eight contacts separated by 2′2 mm. Each contract would have a span of approximately 2 mm. Another example is an electrode with two 1 cm contacts with a 2 mm intervening gap. Yet further, another example of an electrode that can be used in the present methods is a 2 or 3 branched electrode to cover the target site. Each one of these three-pronged electrodes has four 1-2 mm contacts with a center to center separation of 2 of 2.5 mm and a span of 1.5 mm.

The size of each electrode may also vary depending upon such factors as the number of electrodes in the array, the location of the electrodes, the material, the age of the patient, and other factors. In certain aspects, an electrode array has a size (e.g., a diameter) of about 5 mm or less, such as about 4 mm or less, including 4 mm-0.25 mm, 3 mm-0.25 mm, 2 mm-0.25 mm, 1 mm-0.25 mm, or about 3 mm, about 2 mm, about 1 mm, about 0.5 mm, or about 0.25 mm.

In certain embodiments, the method further comprises mapping the epidural space of the subject to optimize positioning of an electrode for applying electrical stimulation. Positioning of an SCS electrode is optimized to maximize clinical responses to electrical stimulation to relieve pain. In some embodiments, the lower thoracic levels, mid-thoracic levels, upper thoracic levels, cervical levels, or other regions of the spine are mapped to determine optimal positioning of SCS electrodes.

Assessment of the effectiveness of electrical stimulation at a particular site for relieving pain may be performed using any standard method. In certain cases, an interviewer-administered pain assessment may be used alone or in conjunction with a self-reporting tool. Interviewer-administered pain assessments may use a visual analog scale or a verbal rating scale. Amelioration of pain may include a reduction in the level of severity of pain compared to the level of pain prior to the spinal cord stimulation. In some embodiments, amelioration of pain includes at least about a 50% to at least about a 90% reduction in the level of pain compared to the level of pain prior to the spinal cord stimulation. In some embodiments, amelioration of pain includes a 50%, 55%, 60%, 65%, 70%, 75%, 80%, 85%, 90%, 95%, or 100% reduction in the level of pain, or any percentage reduction in the level of pain within this range, compared to the level of pain prior to the spinal cord stimulation.

In certain embodiments, the method further comprises mapping the brain of the subject to optimize positioning of a detection electrode. Positioning of the detection electrode is optimized to detect brain activity features that indicate that pain is severe enough to need treatment. For example, the levels of overall power, or power in specific frequency ranges (e.g., alpha, delta, beta, gamma, and/or theta) may be correlated with pain severity and need for treatment with electrical stimulation. In some embodiments, the level of theta frequency power (such as 4 Hz to 8 Hz) and/or beta frequency power (such as 12 Hz to 30 Hz) is correlated with pain severity to determine if a patient is in need of treatment with electrical stimulation. Thus, detection electrodes may be positioned to optimize detection of brain activity in specific frequency ranges that correlate with pain severity of a pain-associated disorder. Alternatively or additionally, coherence within certain spectral frequency bands or other features of network connectivity may be correlated with pain severity and need for treatment with electrical stimulation to the spinal cord.

Detection of brain activity may be performed by any method known in the art. For example, functional brain imaging of neural activity may be carried out by electrical methods such as electroencephalography (EEG), stereoelectroencephalography (sEEG), magnetoencephalography (MEG), single photon emission computed tomography (SPECT), as well as metabolic and blood flow studies such as functional magnetic resonance imaging (fMRI), and positron emission tomography (PET). In some embodiments, the right frontal lobe, left frontal cortex, or other regions are mapped to determine optimal positioning for detection electrodes. One or more of these regions may be implanted with detection electrodes to measure electrical signals from neural activity associated with pain or relief of pain for a subject having a pain-associated disorder.

As set forth here, the subject methods involve applying electrical stimulation to the spinal cord in a manner effective to treat pain caused by a pain-associated disorder in a subject when neural activity associated with pain is detected. Closed-loop therapy can be performed with a spinal cord stimulator used in combination with a neural recording device that records brain electrical signals from neural activity associated with pain, wherein electrical stimulation is delivered to the spinal cord when the level of a brain electrical signal that is detected exceeds a set threshold level indicating a level of pain severity in need of treatment. The parameters for applying the electrical stimulation to the spinal cord may be determined empirically during treatment or may be pre-defined, such as, from a trial study with a subject. For example, brain electrical signal data is recorded (e.g., from the frontal lobe region and/or left frontal cortex) with varying stimulation settings, including baseline (stimulation off), paresthesia threshold, optimal therapeutic stimulation, modified and ineffective stimulation, and maximum tolerated stimulation to identify personal neural signatures of “chronic pain” and “pain relief” for a patient, which are used to assist with spinal cord stimulation programming to determine therapeutic stimulation parameters that achieve analgesia without paresthesia and the minimum stimulation amplitude needed for effective therapy. The parameters of the electrical stimulation may include one or more of frequency, pulse width/duration, duty cycle, intensity/amplitude, pulse pattern, program duration, program frequency, and the like.

Frequency refers to the pulses produced per second during stimulation and is stated in units of Hertz (Hz, e.g., 60 Hz=60 pulses per second). The frequencies of electrical stimulation used in the present methods may vary widely depending on the numerous factors and may be determined empirically during treatment of the subject or may be pre-defined. In certain embodiments, the method may involve applying electrical stimulation to the spinal cord at a frequency of 10 Hz-10 kHz, such as, 10 Hz-500 Hz, 10 Hz-300 Hz, 10 Hz-200 Hz, 10 Hz-150 Hz, 10 Hz-125 Hz, 10 Hz-100 Hz, 10 Hz-50 Hz, 15 Hz-200 Hz, 15 Hz-300 Hz, 20 Hz-200 Hz, 25 Hz-400 Hz, 25 Hz-300 Hz, 25 Hz-200 Hz, 25 Hz-150 Hz, 25 Hz-100 Hz, 50 Hz-500 Hz, 50 Hz-400 Hz, 50 Hz-300 Hz, 50 Hz-200 Hz, 50 Hz-150 Hz, 50 Hz-100 Hz, 75 Hz-300 Hz, 75 Hz-200 Hz, 75 Hz-150 Hz, 75 Hz-125 Hz, 75 Hz-120 Hz, 75 Hz-115 Hz, 75 Hz-110 Hz, or 75 Hz-100 Hz, 1 kHz-10 kHz, 2 kHz-8 kHz, or 4 kHz-6 kHz. The amplitude of current may be 0.1 mA-30 mA, such as, 0.1 mA-25 mA, such as, 0.1 mA-20 mA, 0.1 mA-15 mA, 0.1 mA-10 mA, 1 mA-20 mA, 1 mA-10 mA, 2 mA-30 mA, 2 mA-15 mA, or 2 mA-10 mA.

The electrical stimulation may be applied in pulses such as a uniphasic or a biphasic pulse. The time span of a single pulse is referred to as the pulse width or pulse duration. The pulse width used in the present methods may vary widely depending on numerous factors (e.g., severity of the disease, status of the patient, and the like) and may be determined empirically or may be pre-defined. In certain embodiments, the method may involve applying an electrical stimulation at a pulse width of about 10 μsec-1500 μsec, for example, 30 μsec-1500 μsec, 50 μsec-1500 μsec, 75 μsec-1500 μsec, 100 μsec-1500 μsec, 200 μsec-1500 μsec, 300 μsec-1500 μsec, 500 μsec-1500 μsec, 500 μsec-1000 μsec, 30 μsec-1000 μsec, 50 μsec-1000 μsec, 75 μsec-1000 μsec, 100 μsec-1000 μsec, 200 μsec-1000 μsec, 300 μsec-1000 μsec, 500 μsec-1200 μsec, 30 μsec-500 μsec, 50 μsec-450 μsec, 75 μsec-300 μsec, 100 μsec-200 μsec, or 100 μsec-550 μsec. In some embodiments, the electrical stimulation to the spinal cord is applied at a pulse width of about 10 μsec to about 500 μsec, including any pulse width within this range such as 10, 20, 30, 40, 50, 60, 70, 80, 90, 100, 102, 104, 106, 108, 110, 112, 114, 116, 118, 120, 122, 124, 126, 128, 130, 132, 134, 136, 138, 140, 160, 180, 200, 220, 240, 260, 280, 300, 320, 340, 360, 380, 400, 420, 440, 460, 480, or 500 μsec.

The electrical stimulation may be applied for a stimulation period of 0.1 sec-1 month, with periods of rest (i.e., no electrical stimulation) possible in between. In certain cases, the period of electrical stimulation may be 0.1 sec-1 week, 1 sec-1 day, 10 sec-12 hours, 1 min-6 hours, 10 min-1 hour, and so forth. In certain cases, the period of electrical stimulation may be 1 sec-1 min, 1 sec-30 sec, 1 sec-15 sec, 1 sec-10 sec, 1 sec-6 sec, 1 sec-3 sec, 1 sec-2 sec, or 6 sec-10 sec. The period of rest in between each stimulation period may be 60 sec or less, 30 sec or less, 20 sec or less, or 10 sec.

The electrical stimulation may be applied with an amplitude of current of 0.1 mA-30 mA, such as, 0.1 mA-25 mA, such as, 0.1 mA-20 mA, 0.1 mA-15 mA, 0.1 mA-10 mA, 0.1 mA-2 mA, 0.1 mA-1 mA, 1 mA-20 mA, 1 mA-10 mA, 2 mA-30 mA, 2 mA-15 mA, 2 mA-10 mA, or 1 mA-2 mA. In some embodiments, the amplitude of current is 0.1 mA-1.5 mA, or any amplitude of current in this range such as 0.1, 0.2, 0.3, 0.4, 0.5, 0.6, 0.7, 0.8, 0.9, 1.0, 1.1, 1.2, 1.3, 1.4, or 1.5 mA.

The electrical stimulation having the parameters as set forth above may be applied over a program duration of around 1 day or less, such as, 18 hours, 6 hours, 3 hours, 1 hour, 45 minutes, 30 minutes, 20 minutes, 10 minutes, or 5 minutes, or less, e.g., 1 minute-5 minutes, 2 minutes—10 minutes, 2 minutes—20 minutes, 2 minutes—30 minutes, 5 minutes—10 minutes, 5 minutes—30 minutes, or 5 minutes—15 minutes which period would include the application of pulses and the intervening rest period. The program may be repeated at a desired program frequency to relieve pain in the subject. As such, a treatment regimen may include a program for electrical stimulation at a desired program frequency and program duration. In some embodiments, the treatment regimen is controlled by a control unit in communication with a pulse generator connected to the one or more SCS electrodes in a closed-loop treatment regimen.

In some embodiments, a cap on the maximum number of electrical stimulations per day can be set. For example, the maximum number of electrical stimulations per day may range from 50 therapies per day to 500 therapies per day, including any number of therapies per day in this range such as 50, 75, 100, 125, 150, 175, 200, 225, 250, 275, 300, 325, 350, 375, 400, 425, 450, 475, or 500 therapies per day. Alternatively or additionally a cap can be set on the total amount of time of electrical stimulation per day. For example, the total amount of time of electrical stimulation per day may range from 10 minutes to 100 minutes of total stimulation per day, including any amount of time within this range such as 10, 20, 30, 40, 50, 60, 70, 80, 90, or 100 minutes of total stimulation time per day. Such caps on maximum number of electrical stimulations and/or total amount of time of electrical stimulation per day can be employed so as not to disturb the sleep of a patient from evening therapy. In addition, a rest period without electrical stimulation when a patient wishes to sleep may also be employed to avoid interfering with sleep.

As noted above, the treatment may ameliorate pain of a pain-associated disorder suffered by the subject. Assessment of effectiveness of the treatment may be performed using any known method for evaluating pain. In certain cases, an interviewer-administered pain assessment may be used alone or in conjunction with a self-reporting tool. Interviewer-administered pain assessments may use a visual analog scale or a verbal rating scale. Amelioration of pain may include a reduction in the level of severity of pain compared to the level of pain prior to the spinal cord stimulation.

In certain cases, effectiveness of treatment may be assessed by detecting activity (e.g., electrical signals) associated with pain or pain relief, which may be within the right frontal lobe, left frontal cortex, or another area. For example, the brain region(s) may be one or more of the right frontal lobe and left frontal cortex regions. Detection of brain activity may be performed by functional brain imaging. Functional brain imaging may be carried out by electrical methods such as electroencephalography (EEG), magnetoencephalography (MEG), single photon emission computed tomography (SPECT), as well as metabolic and blood flow studies such as functional magnetic resonance imaging (fMRI), and positron emission tomography (PET). In some embodiments, electrical methods for assessing effectiveness of treatment may involve use of a detection electrode as described herein or placement of an additional electrode for measuring electrical signals at a secondary region of the brain. One or more regions of the brain may be implanted with an electrode and electrical signals measured for assessment of effectiveness of the treatment. Any suitable electrodes may be used for measurements and may include one or more surface electrodes (non-brain penetrating electrode(s)) or one or more depth electrodes (brain penetrating electrode(s)) as described herein.

Assessment of effectiveness of treatment and assessment of amelioration of pain of a pain-associated disorder may be performed at any suitable time point after commencement of the treatment procedure, for example, during open-loop or closed-loop therapy or after a treatment regimen is complete. Embodiments of the subject methods include assessing effectiveness of treatment or amelioration of pain from the pain-associated disorder within seconds, minutes, hours, or days after the initial treatment regimen has been completed. In some instances, assessment may be performed at multiple time points. In some cases, more than one type of assessment may be performed at the different time points. In some embodiments, a subject's brain activity (e.g., at the right frontal lobe and/or left frontal cortex) may be measured prior to the application of electrical stimulation, and assessing may include comparing the subject's brain activity after the treatment to that before the treatment and a change in the post-treatment brain activity may indicate successful treatment.

In some embodiments, the subject is monitored for a period of time during open-loop or closed-loop therapy using a detection electrode, wherein increasing numbers of the brain electrical signals exceeding the threshold level detected by the detection electrode within a later recording period compared to an earlier recording period indicate increasing severity of pain from the pain-associated disorder; and decreasing numbers of brain electrical signals exceeding the threshold level detected by the detection electrode within a later recording period compared to an earlier recording period indicate decreasing severity of the pain of the pain-associated disorder.

The methods and systems provided herein may be used to ameliorate pain in a patient. A person skilled in the art will appreciate that amelioration of pain may provide relief to a patient suffering from two separate pain-associated disorders.

Upon completion of a treatment regimen, the patient may be assessed for effectiveness of the treatment and the treatment regimen may be repeated, if needed. In certain cases, the treatment regimen may be altered before repeating. For example, one or more of the frequency, pulse width, current amplitude, period of electrical stimulation, program duration, program frequency, and/or placement of SCS or detection electrodes may be altered before starting a second treatment regimen.

Application of the method may include a prior step of selecting a patient for treatment based on need as determined by clinical assessment, which may include assessment of severity of chronic pain (e.g., pain lasting at least 3 months), physical condition, medication regime, pain history, cognitive assessment, anatomical assessment, behavioral assessment and/or neurophysiological assessment. In certain cases, a subject may be further assessed to determine if spinal cord stimulation will completely or partially (e.g., at least 50%) relieve the chronic pain. Such a patient may undergo SCS on a temporary trial basis to determine if SCS decreases the severity of pain experienced by the patient. Such a patient may also be implanted with detection electrodes to identify personalized pain and pain relief neural signatures of “chronic pain” and “pain relief” to assist with spinal cord stimulation programming to determine therapeutic stimulation parameters for the patient and/or evaluate whether SCS therapy will be effective for ameliorating pain for the patient.

In certain aspects, the methods and systems of the present disclosure may include measurement of brain activity, for example, electrical activity in the right frontal lobe and/or the left frontal cortex, where the level of theta and/or beta frequency power may be measured. In certain cases, electrical activity from a plurality of locations in the right frontal lobe and/or the left frontal cortex may be measured and averaged. In some embodiments, electrical activity in the theta frequency range (such as 4 Hz to 8 Hz) and/or beta frequency range (such as 12 Hz to 30 Hz) may be measured from the right frontal lobe and/or the left frontal cortex. In some cases, electrical activity in one or more locations in the brain may be measured during a period extending from prior to stimulation to the period during which stimulation to the spinal cord is applied, or to a period after stimulation to the spinal cord has been applied, and monitored for a decrease in the power of theta frequency (such as 4 Hz to 8 Hz) and/or beta frequency (such as 12 Hz to 30 Hz) activity. In some cases, when the power of theta frequency (such as 4 Hz to 8 Hz) and/or beta frequency (such as 12 Hz to 30 Hz) activity is within a normal range (e.g., a range associated with a substantial lack of pain), the methods and systems do not apply a further stimulation to the spinal cord. Alternatively, when the power of theta frequency (such as 4 Hz to 8 Hz) and/or beta frequency (such as 12 Hz to 30 Hz) activity is not within a normal range (e.g., a range associated with substantial pain), the methods and systems may apply a further stimulation to the spinal cord. In certain cases, the application of electrical stimulation to the spinal cord may suppress theta frequency (such as 4 Hz to 8 Hz) and/or beta frequency (such as 12 Hz to 30 Hz) activity across the right frontal lobe and/or the left frontal cortex. The decrease may be as compared to the power prior to the application of stimulation. In certain cases, the application of electrical stimulation to the spinal cord may alter other neural features from one more regions of the brain. The alterations may be compared to the state of these features prior to the application of stimulation.

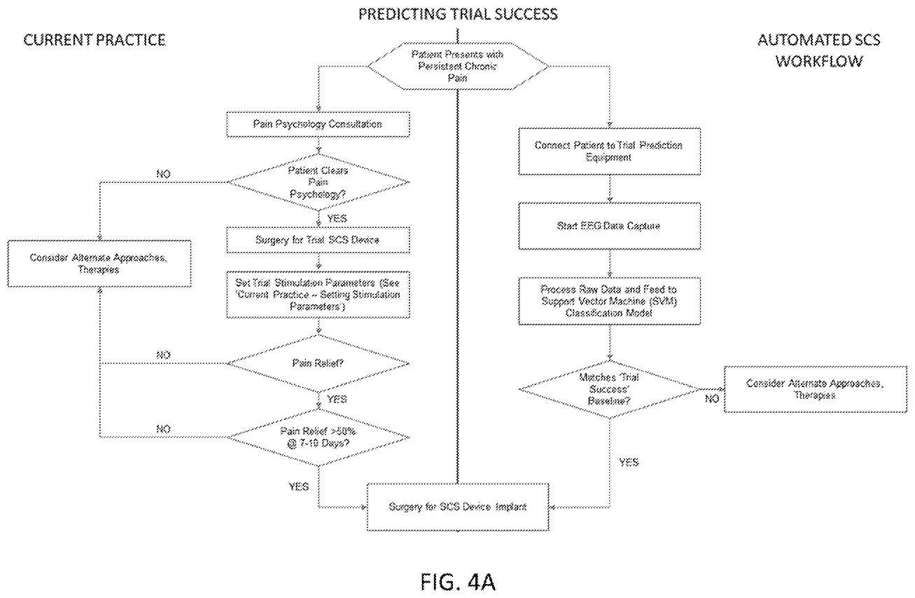

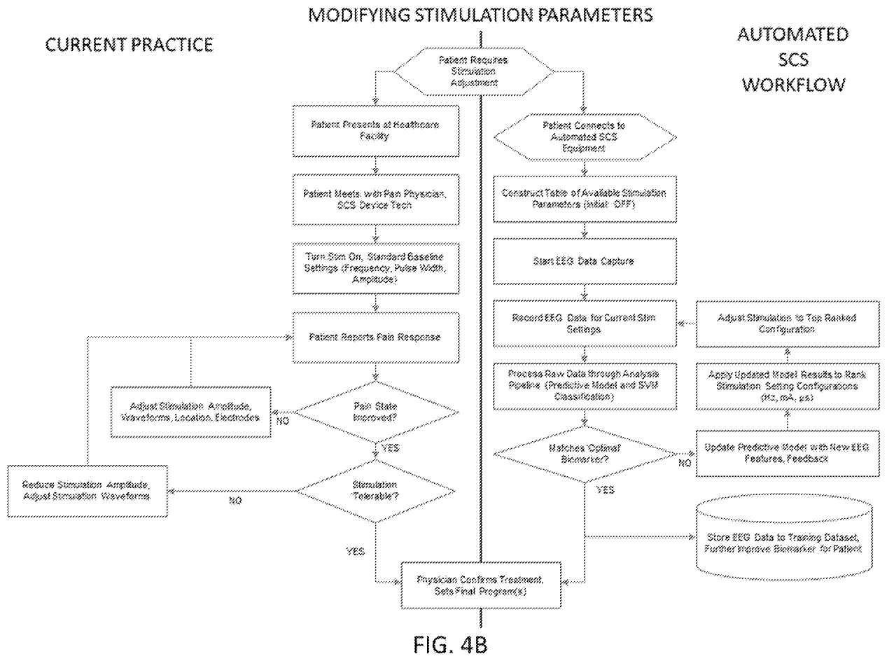

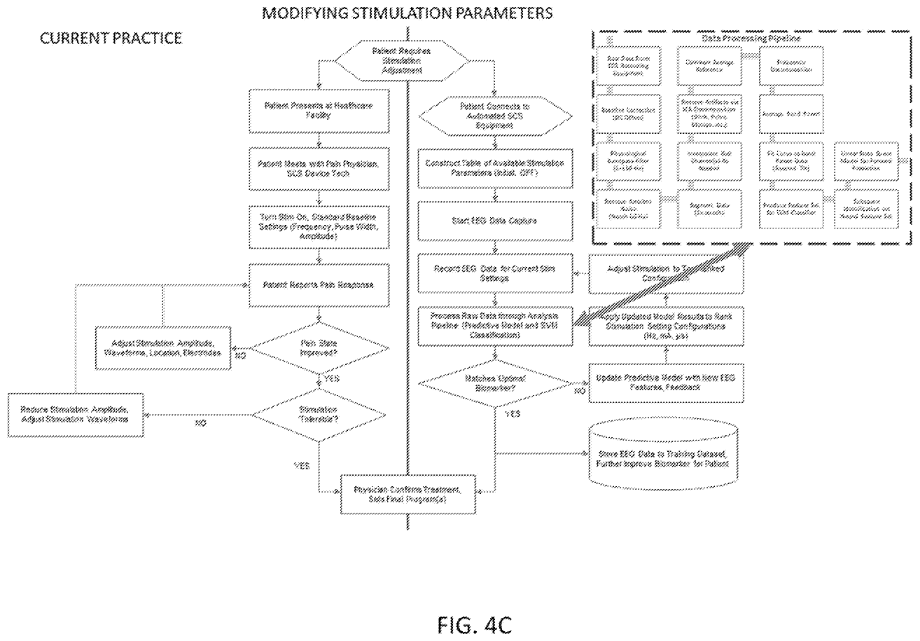

A closed-loop method allows determination of parameters of electrical stimulation based upon real-time feedback signals from the brain of the subject. Closed-loop methods and systems allow for automation of treatment of the subject including real-time need-based modulation of the treatment regimen. Exemplary closed-loop methods and associated systems for treatment of a neuropsychiatric disorder are further discussed in the Examples section and are depicted in FIGS. 4B and 4C. Closed-loop methods and systems for automated delivery of electrical stimulation are further described below.

### Closed-Loop Method for Automated Delivery of Electrical Stimulation

In certain embodiments, a control algorithm is used to automate the delivery of electrical stimulation to the spinal cord in response to detection of neural activity associated with pain. According to certain embodiments, the method may include receiving an electrical signal from a region (e.g., right frontal lobe and/or the left frontal cortex) of the brain of the subject via a detection electrode; applying electrical signal metrics to a control algorithm that is tuned to a clinically relevant target (e.g., a range of signal indicative of effective treatment and/or a range of signal indicative of severity of pain and the need for treatment); automatically delivering electrical stimulation to the spinal cord via an SCS electrode in a manner effective to treat the pain if the electrical signal metrics indicate that the patient is in need of treatment. For example, electrical activity in the theta frequency range (such as 4 Hz to 8 Hz) and/or beta frequency range (such as 12 Hz to 30 Hz) from the right frontal lobe and/or the left frontal cortex may be measured with a detection electrode, wherein the control algorithm receives the electrical activity data from the detection electrode and automates delivery of electrical stimulation via an SCS electrode to the spinal cord when the level of theta frequency (such as 4 Hz to 8 Hz) and/or beta frequency (such as 12 Hz to 30 Hz) power is in a certain range indicating that a patient has pain severe enough that the patient is in need of treatment. In some embodiments, one or more programmed stimulation parameters are modulated according to the algorithm's control law based on the recorded electrical activity data; and modulated electrical stimulation is delivered to the spinal cord via the SCS electrode in a manner effective to ameliorate the pain.

As described in the foregoing sections, effectiveness of treatment or amelioration of pain from a pain-associated disorder may be assessed by detecting brain electrical activity associated with pain using a detection electrode. In an open-loop system, stimulation is delivered in a pre-programmed way or manually by a user but is not automatically controlled by real-time neural feedback from the patient's brain. The electrical activity may be analyzed by a computing means which may output recommendations based on comparing the electrical activity to a predetermined range. A user may then carry out the recommendations, such as, changing a parameter of the electrical stimulation program prior to starting another treatment regimen. In a closed-loop system, by contrast, a computing means can automatically update stimulation parameters based upon analysis of the recorded electrical signal and/or automatically deliver stimulation to the spinal cord according to the electrical stimulation program. In some embodiments, either an open-loop or a closed-loop system may be integrated with a mechanism for user intervention, for example by allowing user-override of open-loop or closed-loop stimulation programs to enact or prevent stimulation that would ordinarily occur, or to manually change parameters of such stimulation.

In some embodiments, the computing means for directing closed-loop stimulation may be a combination of hardware/software which may be connected wirelessly or by wire to the measurement electrodes. The computing means may communicate with a control unit (also referred to as a control module) that controls a pulse generator connected to the SCS electrodes. In certain embodiments, the computing means may be connected to a recorder (e.g., a neurophysiological recorder or neural recording device) that records brain activity measured by the detection electrodes. The computing means may include a control algorithm that determines modification of stimulation parameters based on real-time outputs of the neurophysiological recorder. The algorithm may operate by simple on/off control of stimulation at set parameters, modifying only the on/off parameter with each evaluation cycle, or may determine sophisticated modification of a range of stimulation parameters with each cycle. In some cases, the algorithm may be based on information related to the pain-associated disorder, such as, a range of electrical activity that is indicative of the presence of pain. The algorithm may also include additional information such as a brain activity profile of a normal subject (not suffering from pain). Regardless of the particular control algorithm structure, the computing means may be tuned to a clinically relevant target (e.g., a range of signal indicative of effective treatment and/or a range of signal indicative of severity of pain and the need for treatment) that directs modulation of one or more programmed stimulation parameters according to the algorithm's control law, applying the modulated electrical stimulation to the spinal cord via the SCS electrode.

In some cases, the computing means, via a control algorithm, may determine whether the received electrical signals are within or outside a predetermined range of neural signals indicative of the presence of pain. When the received electrical signals are outside this predetermined range, then the computing means determines that the pain has been treated or ameliorated. The computing means may then communicate with the control unit to direct stimulation shut-off by the pulse generator. When the received electrical signals are within the predetermined range of neural signals indicative of the presence of the pain, then the computing means determines that the pain is in need of treatment and/or severe enough to require treatment. The control algorithm within the computing means may then determine whether the initial step of applying electrical stimulation to the spinal cord should be repeated and/or whether a parameter of the electrical stimulation should be modified prior to the step of applying electrical stimulation. The computing means, via the control unit, may then communicate with the control unit to provide the appropriate instructions to the pulse generator.

In some embodiments, the computing means may determine whether the received electrical signals are within or outside a second predetermined range, where the second predetermined range is indicative of treatment and/or amelioration of pain from the pains-associated disorder. When the received electrical signals are within the second predetermined range, then the computing means determines that the pain has been treated and/or ameliorated. The computing means may then communicate with the control unit to direct stimulation switch-off by the pulse generator. When the received electrical signals are outside the second predetermined range, then the computing means determines that the pain still needs to be treated and/or ameliorated. The control algorithm within the computing means may then determine whether the initial step of applying electrical stimulation should be repeated and/or whether a parameter of the electrical stimulation modified prior to the step of applying electrical stimulation. The processor may then communicate with the control unit to provide the appropriate instructions to the pulse generator.

Thus, in certain aspects, the subject methods operate as a closed-loop control system which may automatically adjust one or more parameters in response to electrical activity from a region of the brain of a subject and/or automatically deliver stimulation to the spinal cord according to the electrical stimulation program. In some embodiments, the closed-loop control system automatically delivers stimulation according to set parameters when the received electrical signals are within a predetermined range indicative of the need for treatment. Exemplary closed-loop methods and associated systems are described in the Examples section of the application and are illustrated in FIGS. 4A-4C.

In some aspects, the closed loop system may be used to sense a subject's need for treatment using the methods disclosed herein. For example, the closed loop system may be programmed to monitor brain activity from one or more regions of the brain and compare the brain activity to a range indicative of pain severity. Upon detection of electrical activity indicative of pain, the closed loop system may automatically commence a treatment protocol of applying electrical stimulation to the spinal cord.

In additional aspects, the closed loop system may be used as a system for monitoring brain activity and correlating the brain activity to the subject's level of pain. For example, since the closed loop system is configured for recording electrical signals from a subject's brain, the subject's level of pain may be monitored in real-time and correlated to the measured electrical signals to provide a biomarker that is related to the subject's pain. For example, electrical activity measured when a subject is experiencing pain can be used to develop a biomarker, e.g., as range of electrical activity indicative of pain, and so on. As such, closed loop systems are useful for detecting pain caused by pain-associated disorders.

It is understood that electrical signals that are indicative of pain or pain relief for a subject may be recorded from a subject's brain and may be used in aspects outside of a closed loop system. For example, electrical signals indicative of pain or pain relief for a subject may be recorded from the right frontal lobe, left frontal cortex, or other brain region using electrodes or another device operably coupled to the patient's brain, which electrodes or device may or may not be part of a closed loop system. The patient may be treated as disclosed herein (e.g., by applying electrical stimulation to the spinal cord), and electrical signals recorded from the right frontal lobe, left frontal cortex, or other region in real time as the treatment is administered or after the treatment is administered. The electric signals recorded after the administration of electrical stimulation is commenced may then be compared to the electric signals recorded prior to the treatment to determine features in the recorded electric signals that change post-treatment. These features provide a feedback signal to indicate whether the treatment is having an effect on the patient's level of pain. These features can also serve as feedback signals to a closed loop system. These features may include the overall power, or power in specific frequency ranges (e.g. alpha, delta, beta, gamma, and/or theta). In some cases, these features may be patient specific or specific to a particular pain state or both. For example, some of the features may be features found in a plurality of patients having a particular level of pain; some of the features may be features in a particular patient which may not be found in a significant number of other patients having the same level of pain. In some embodiments, a combination of patient-specific features and pain-specific features may be monitored to assess efficacy of treatment.

In a particular aspect, the closed loop system and methods provided herein may involve a recording of electrical signals from one or more regions (e.g., right frontal lobe, left frontal cortex, or other region) of a patient's brain, where the patient has a pain-associated disorder. The patient may then be treated by application of electrical stimulation to the spinal cod, and electrical signals may be recorded from the same regions of the brain (e.g., right frontal lobe, left frontal cortex, or other region) and compared to the pre-treatment recording. Features in the recorded signals that changed after the treatment would correspond to biomarkers that indicate whether the treatment is having an effect. The change in recorded signals can also optionally be correlated to the level of pain reported by the patient after the treatment. The change can be used for modulating the treatment in a closed loop system. For example, when the change in the recorded signal correlates with amelioration of pain, those features would indicate to a computing means of a closed loop system that further treatment need not be performed.

In certain aspects, methods of the present disclosure that may be embodied in an open- or a closed-loop system may include measuring brain activity from a subject having chronic pain, when the subject has a level of theta frequency (such as 4 Hz to 8 Hz) and/or beta frequency (such as 12 Hz to 30 Hz) brain activity that is higher than a normal range (e.g., range reflective of a normal brain, such as, brain of a person not suffering from pain) or higher than a threshold level (e.g., indicating pain severe enough that treatment is needed), the subject is treated by applying electrical stimulation to the spinal cord via an SCS electrode positioned in the epidural space in a manner effective to reduce the level of theta or beta range frequency activity in the brain of the subject. The brain activity may be measured at one or more brain regions (e.g., right frontal lobe, left frontal cortex, or other region). In some aspects, the system may monitor brain activity such as levels of theta and/or beta frequency activity, and if the level of theta and/or beta frequency activity is higher than a reference range (e.g., range reflective of a normal brain, such as, brain of a person not suffering from pain) and/or higher than a threshold level (e.g., indicating pain severe enough that treatment is needed), the closed loop system may apply electrical stimulation to the spinal cord via an electrode positioned in the epidural space in a manner effective to reduce the level of theta frequency (such as 4 Hz to 8 Hz) and/or beta frequency (such as 12 Hz to 30 Hz) activity in the brain of the subject.

In some embodiments, one or more pattern recognition methods can be used in analyzing recorded brain electrical activity data to automate detection of brain activity features that distinguish pain states that are severe enough to need treatment from symptomless states (baseline) or minor pain states that do not need treatment. The models and/or algorithms can be provided in machine readable format and may be used to correlate the levels of overall power, or power in specific frequency ranges (e.g., alpha, delta, beta, gamma, and/or theta) with pain severity and need for treatment with electrical stimulation to the spinal cord. In some embodiments, the level of theta frequency (such as 4 Hz to 8 Hz) and/or beta frequency (such as 12 Hz to 30 Hz) power is correlated with pain severity to determine if a patient is in need of treatment with electrical stimulation. Alternatively or additionally, coherence within certain spectral frequency bands or other features of network connectivity may be correlated with pain severity and need for treatment with electrical stimulation to the spinal cord.

In some embodiments, frequency decompositions are used to calculate the average power in each neurophysiological activity band (e.g., delta, theta, alpha, beta, gamma etc.). The slope of 1/f power values across frequency bands is determined for a given epoch by fitting a curve to the band power data for that epoch (e.g., slope is calculated on multiple samples for each EEG contact and each time epoch, over the axes ‘frequency band’ vs ‘power’); this value is referred to as the spectral tilt. The spectral tilt values are fit to a pain classification model to determine how to adjust the programmed stimulation parameters. See, e.g., Example 5.

In some embodiments, a computer implemented method is used for selecting stimulation settings, the computer performing steps comprising a) ranking predicted stimulation effectiveness for the available settings of a spinal cord stimulator based on classifier scores for stimulation effectiveness of each setting using a linear classification model; b) selecting stimulation settings predicted to have the highest stimulation effectiveness based on the linear classification model; c) receiving recorded brain electrical signal data from the frontal lobe region of the brain of the subject after applying electrical stimulation with the spinal cord stimulator to the spinal cord using the settings predicted to have the highest stimulation effectiveness; d) analyzing the recorded brain electrical signal data to evaluate the neural response of the subject to the electrical stimulation; e) updating the linear classification model based on the neural response of the subject to the electrical stimulation to generate an updated linear classification model; f) updating the ranking of predicted stimulation effectiveness for the available settings of the spinal cord stimulator using the updated linear classification model; g) selecting stimulation settings predicted to have the highest stimulation effectiveness based on the updated linear classification model; h) receiving recorded brain electrical signal data from the frontal lobe region of the brain of the subject after applying the electrical stimulation with the spinal cord stimulator to the spinal cord using the settings predicted to have highest stimulation effectiveness based on the updated linear classification model; and repeating e)-h) to adjust the available settings of the spinal cord stimulator to optimize stimulation effectiveness. See, e.g., Example 6.

In some embodiments, a computer implemented method is used to predict whether a subject who has chronic pain will respond to spinal cord stimulation based on calculating a classifier score to predict the likelihood of SCS therapy success. See, e.g., Example 7.

Analyzing the recorded brain electrical activity may comprise the use of an algorithm or classifier. In some embodiments, a machine learning algorithm is used to classify brain activity as indicating whether or not pain is severe enough that electrical stimulation should be delivered to the spinal cord of the patient. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines (SVM), Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network (recurrent or convoluted), Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing. The methods described herein can be implemented in digital electronic circuitry, or in computer software, firmware, or hardware.

The disclosed and other embodiments can be implemented as one or more computer program products, i.e., one or more modules of computer program instructions encoded on a computer readable medium for execution by, or to control the operation of, a data processing apparatus. The computer readable medium can be a machine-readable storage device, a machine-readable storage substrate, a memory device, a composition of matter effecting a machine-readable propagated signal, or any combination thereof.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

In a further aspect, the system for performing the computer implemented method, as described, may include a computer containing a processor, a storage component (i.e., memory), a display component, and other components typically present in general purpose computers. The storage component stores information accessible by the processor, including instructions that may be executed by the processor and data that may be retrieved, manipulated or stored by the processor.

The storage component may be of any type capable of storing information accessible by the processor, such as a hard-drive, memory card, ROM, RAM, DVD, CD-ROM, USB Flash drive, write-capable, and read-only memories. The processor may be any well-known processor, such as processors from Intel Corporation. Alternatively, the processor may be a dedicated controller such as an ASIC.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data.

In certain embodiments, the processor and storage component may comprise multiple processors and storage components that may or may not be stored within the same physical housing. For example, some of the instructions and data may be stored on removable CD-ROM and others within a read-only computer chip. Some or all of the instructions and data may be stored in a location physically remote from, yet still accessible by, the processor. Similarly, the processor may comprise a collection of processors which may or may not operate in parallel. In some embodiments, a hardware accelerator is used.

In some embodiments, the method is performed using a cloud computing system. In these embodiments, the data files and the programming can be exported to a cloud computer, which runs the program, and returns an output to the user.

Components of systems for carrying out the presently disclosed methods are further described in the examples below.

### Administration of a Pharmacological Agent

Embodiments of the methods and systems provided in this disclosure may also include administration of an effective amount of at least one pharmacological agent. By “effective amount” is meant a dosage sufficient to prevent or treat a pain-associated disorder in a subject as desired. The effective amount will vary somewhat from subject to subject, and may depend upon factors such as the age and physical condition of the subject, severity of the pain-associated disorder being treated, the duration of the treatment, the nature of any concurrent treatment, the form of the agent, the pharmaceutically acceptable carrier used if any, the route and method of delivery, and analogous factors within the knowledge and expertise of those skilled in the art. Appropriate dosages may be determined in accordance with routine pharmacological procedures known to those skilled in the art, as described in greater detail below.

If a pharmacological approach is employed in the treatment of a pain-associated disorder, the specific nature and dosing schedule of the agent will vary depending on the particular nature of the disorder to be treated. Representative pharmacological agents that may find use in certain embodiments of the subject invention include, but are not limited to, nonsteroidal anti-inflammatory drugs (NSAIDs) such as aspirin, ibuprofen and naproxen; COX2 inhibitors such as rofecoxib, celecoxib, and etoricoxib; opioids such as morphine, codeine, oxycodone, hydrocodone, dihydromorphine, and pethidine; selective serotonin reuptake inhibitors (SSRIs) such as fluoxetine (Prozac), paroxetine (Paxil, Pexeva), sertraline (Zoloft), citalopram (Celexa) and escitalopram (Lexapro); and serotonin-norepinephrine reuptake inhibitors (SNRIs) such as duloxetine (Cymbalta), venlafaxine (Effexor XR), desvenlafaxine (Pristiq, Khedezla) and levomilnacipran (Fetzima), and the like.

In certain aspects, the administration of a pharmacological agent involves using a pharmacological delivery device such as, but not limited to, pumps (implantable or external devices), epidural injectors, syringes or other injection apparatus, catheter and/or reservoir operatively associated with a catheter, etc. For example, in certain embodiments a delivery device employed to deliver at least one pharmacological agent to a subject may be a pump, syringe, catheter or reservoir operably associated with a connecting device such as a catheter, tubing, or the like. Containers suitable for delivery of at least one pharmacological agent to a pharmacological agent administration device include instruments of containment that may be used to deliver, place, attach, and/or insert the at least one pharmacological agent into the delivery device for administration of the pharmacological agent to a subject and include, but are not limited to, vials, ampules, tubes, capsules, bottles, syringes and bags. Administration of a pharmacological agent may be performed by a user or by a closed loop system.

### Systems

The present disclosure also provides systems which find use, e.g., in practicing the subject methods. The system may be an open-loop or closed-loop system configured for performing the methods provided herein. In some embodiments, the system may include a SCS electrode adapted for positioning in the epidural space (e.g., at the lower thoracic level, mid-thoracic level, upper thoracic level, cervical level, or other region of the spine) of the subject and a detection electrode adapted for positioning at a brain region for detection of neural activity associated with pain or pain relief (e.g., right frontal lobe, left frontal cortex, or other region) and for recording an electrical signal from the brain region before, during, or after an electrical stimulation is applied to the spinal cord. In a closed-loop system, the system may also include a computing means and control unit programmed to instruct the SCS electrode to apply an electrical stimulation to the spinal cord in a manner effective to treat the pain in the subject and/or ameliorate the pan caused by the pain-associated disorder; receive the electrical signals associated with pain or pain relief from the brain of the subject via the detection electrode; apply electrical signal metrics to a control algorithm that is tuned to a clinically relevant target (e.g., a range of signal indicative of severity of pain and the need for treatment); and automatically delivering electrical stimulation to the spinal cord via the control unit, pulse generator and SCS electrode in a manner effective to treat the pain or ameliorate the pain of the pain-associated disorder if the electrical signal metrics indicate that the patient is in need of treatment. For example, electrical activity in the theta frequency range (such as 4 Hz to 8 Hz) and/or beta frequency range (such as 12 Hz to 30 Hz) from the right amygdala, left amygdala, right orbitofrontal cortex, left subgenual cingulate, or right hippocampus, or other region may be measured with a detection electrode using this system, wherein the control algorithm receives the electrical activity data from the detection electrode and automates delivery of electrical stimulation via the control unit, a pulse generator and the SCS electrode to the spinal cord when the level of theta frequency (such as 4 Hz to 8 Hz) and/or beta frequency (such as 12 Hz to 30 Hz) power is in a certain range indicating that a patient has pain severe enough that the patient is in need of treatment. In some embodiments, one or more programmed stimulation parameters are modulated according to the algorithm's control law based on the recorded electrical activity data, and modulated electrical stimulation is delivered to the spinal cord via the control unit, pulse generator and SCS electrode in a manner effective to ameliorate pain caused by the pain-associated disorder. The closed loop system may include an on-body pulse generator that is connected to the implanted SCS electrodes and hence can apply electrical stimulation to the spinal cord automatically upon receiving a communication from the control unit.

The processor of the closed-loop system may run programming for assessing the effectiveness of treatment and/or effectiveness of amelioration of pain and modulate a parameter of the treatment as needed without user intervention. Thus, the closed-loop system may not necessarily include a user interface for a user to instruct the SCS electrode to apply an electrical stimulation to the spinal cord to treat the pain in the subject. However, in some embodiments, a user interface may be included in the closed-loop system which may be used to confirm the recommendation of the closed loop system or to override it or to change the recommendation.

In certain aspects, a control algorithm for the methods and systems of the present disclosure may include steps of comparing an electrical signal from a region of the brain of a subject to a normal or reference electrical signal (e.g., for substantially pain-free state), wherein when the electrical signal is significantly different from the normal or reference electrical signal, the control algorithm includes steps of directing a device to apply electrical stimulation to the spinal cord of the subject, followed by measurement of electrical signals from the region of the brain and comparing it to a normal or reference electrical signal, where when the measured signal is significantly different from normal or reference electrical signal, the algorithm includes the step of applying another electrical stimulation to the spinal cord.

In some embodiments, the control algorithm utilizes a machine learning algorithm to analyze inputted brain electrical activity data to automate detection of brain activity features that distinguish pain states that are severe enough to need treatment from symptomless states (baseline) or minor pain states that do not need treatment. The control algorithm then directs a device to apply electrical stimulation to the spinal cord of the subject if the brain activity features indicate a pain state severe enough that the subject should be treated with electrical stimulation. For example, a machine learning algorithm may be used to correlate the levels of overall power, or power in specific frequency ranges (e.g., alpha, delta, beta, gamma, and/or theta) with pain severity and need for treatment with electrical stimulation to the spinal cord. In some embodiments, the level of theta frequency (such as 4 Hz to 8 Hz) and/or beta frequency (such as 12 Hz to 30 Hz) power is correlated with pain severity to determine if a patient is in need of treatment with electrical stimulation. In some embodiments, electrical stimulation is delivered to the spinal cord when the level of an electrical signal that is detected exceeds a set threshold level. In some embodiments, spectral tilt values are fit to a pain classification model to determine how to adjust one or more programmed stimulation parameters. In certain embodiments the algorithm provides updated optimal stimulation setting recommendations to the clinician for guiding programing and decision making.

Components of systems for carrying out the presently disclosed methods are further described in the examples below.

### Utility

The methods and systems of the present disclosure find use in a variety of different applications, including the treatment of pain such as chronic, idiopathic, nociceptive or neuropathic pain. The pain may be caused by a pain associated disorder such as, but not limited to, back pain, failed back surgery syndrome, spinal cord injury, spinal stenosis, post-surgical pain, complex regional pain syndrome, arachnoiditis, angina, nerve-related pain (e.g., such as caused by diabetic neuropathy, cancer-related neuropathy, or nerve damage caused by radiation, surgery, or chemotherapy), peripheral vascular disease, pain after an amputation, visceral abdominal pain, perineal pain, multiple sclerosis, arthritis, or chronic leg pain (e.g., sciatica), neck pain, foot pain, or arm pain.

In certain aspects, the methods and systems of the present disclosure may be used to treat chronic pain as well other symptoms associated with chronic pain, e.g., constant attention to the experience of pain, emotional toll of the experience of constant pain, fatigue, depression and/or anxiety. Efficacy of the treatment of patients suffering from chronic pain may be measured in an art accepted manner such as, by using a visual analog scale or a verbal rating scale.

Closed-loop stimulation can be finely targeted and tuned in a personalized manner to achieve more reliable and/or more effective pain relief compared to convention SCS techniques. Likewise, these same attributes of targeting and tuning are more likely to impart lasting relief of pain, given the ability to find optimal SCS programming parameters that deliver analgesia without paresthesia and deliver SCS therapy at the minimum stimulation amplitude necessary to relieve pain.

Using recorded brain activity data including neural signatures indicative of pain and/or pain relief to guide programming sessions during initial SCS programming enables clinicians to efficiently and objectively determine a patient's ideal stimulation settings, thereby reducing the duration and increasing the success rate of the trial process. Additionally, finding the stimulation amplitude that provides pain relief without paresthesia reduces the overall electrical dosage delivered during therapy. This reduction in electrical exposure delays loss of efficacy due to nervous system adaptation, as well as extends the battery life of the implanted pulse generator to improve the time between recharges and/or replacements.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-70 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A method for treating chronic pain in a subject, the method comprising:
   positioning a first electrode at a location in the epidural space to deliver electrical stimulation to the spinal cord of the subject;
   positioning a second electrode at a location in a frontal lobe region of the brain of the subject to detect a brain electrical signal associated with the chronic pain;
   detecting the brain electrical signal at the frontal lobe region of the brain of the subject using the second electrode; and
   applying electrical stimulation to the spinal cord using the first electrode in a manner effective to treat the chronic pain in the subject when the brain electrical signal detected using the second electrode exceeds a threshold level.
2. The method of aspect 1, further comprising using a control algorithm to automate said applying electrical stimulation when the brain electrical signal exceeds a threshold level.
3. The method of aspect 2, wherein the control algorithm uses a machine learning algorithm for pain classification.
4. The method of aspect 3, wherein the machine learning algorithm is a supervised machine learning algorithm.
5. The method of aspect 4, wherein the supervised machine learning algorithm is a support vector machine (SVM) learning algorithm.
6. The method of any one of aspects 2-5, wherein the control algorithm further modulates one or more programmed stimulation parameters based on a level of power of the brain electrical signal.
7. The method of any one of aspects 2-6, wherein the control algorithm further determines the minimum stimulation amplitude needed to relieve the chronic pain based on a level of power of the brain electrical signal.
8. The method of any one of aspects 1-7, wherein said applying the electrical stimulation comprises applying the electrical stimulation to the spinal cord at the minimum stimulation amplitude needed to relieve the chronic pain.
9. The method of any one of aspects 1-8, further comprising positioning a plurality of electrodes at the location in the frontal lobe region of the brain of the subject for detection of the brain electrical signal by stereoelectroencephalography (sEEG).
10. The method of any one of aspects 1-9, wherein the frontal lobe region is a right frontal lobe region of the brain.
11. The method of any one of aspects 1-10, wherein the electrical stimulation is applied unilaterally or bilaterally.
12. The method of any one of aspects 1-11, wherein the brain electrical signal comprises alpha frequency, beta frequency, gamma frequency, delta frequency, or theta frequency neural oscillations.
13. The method of aspect 12, wherein the theta frequency neural oscillations are in a range from 4 Hz to 8 Hz.
14. The method of aspect 12, wherein the beta frequency neural oscillations are in a range from 12 Hz to 30 Hz.
15. The method of any one of aspects 1-14, wherein the second electrode is placed on a surface of a right frontal lobe region or within a right frontal lobe region.
16. The method of any one of aspects 1-15, wherein the second electrode is a non-brain penetrating surface electrode array or a brain-penetrating electrode array.
17. The method of any one of aspects 1-16, further comprising positioning a third electrode at a location in a left frontal cortex region of the brain of the subject to detect a brain electrical signal associated with relief of the chronic pain.
18. The method of aspect 17, wherein the third electrode is placed on a surface of the left frontal cortex region or within the left frontal cortex region.
19. The method of aspect 17 or 18, wherein the third electrode is a non-brain penetrating surface electrode array or a brain-penetrating electrode array.
20. The method of any one of aspects 17-19, further comprising using a control algorithm to automate adjustment of one or more programmed stimulation parameters to maintain the level of the brain electrical signal associated with relief of the chronic pain in a target range.
21. The method of any one of aspects 1-20, further comprising determining a paresthesia threshold for the electrical stimulation; and using a control algorithm to automate adjustment of one or more programmed stimulation parameters to apply the electrical stimulation at a level below the paresthesia threshold.
22. The method of any one of aspects 1-21, wherein the chronic pain is caused by a pain-associated disorder, wherein applying the electrical stimulation relieves the pain.
23. The method of aspect 22, wherein the pain-associated disorder is back pain, failed back surgery syndrome, spinal cord injury, spinal stenosis, post-surgical pain, complex regional pain syndrome, arachnoiditis, angina, nerve-related pain, peripheral vascular disease, pain after an amputation, visceral abdominal pain, perineal pain, multiple sclerosis, arthritis, or chronic leg pain, neck pain, foot pain, or arm pain.
24. The method of aspect 23, wherein the nerve-related pain is caused by diabetic neuropathy, cancer-related neuropathy, or nerve damage caused by radiation, surgery, or chemotherapy.
25. The method of any one of aspects 1-24, wherein the method further comprises assessing effectiveness of the treatment in the subject.
26. The method of any one of aspects 1-25, further comprising mapping the brain of the subject to identify an optimal location in the right frontal lobe region to detect the brain electrical signal associated with the chronic pain.
27. The method of any one of aspects 1-26, further comprising mapping the brain of the subject to identify an optimal location in the left frontal cortex region to detect the brain electrical signal associated with relief of the chronic pain.
28. The method of any one of aspects 1-28, further comprising assessing relief of pain during or after treatment of the subject by using a visual analog scale or a verbal rating scale.
29. The method of any one of aspects 1-28, further comprising repositioning the first electrode in the epidural space to improve relief of pain.
30. The method of any one of aspects 1-29, wherein the location in the epidural space is in a lower thoracic level, a mid-thoracic level, an upper thoracic level, or a cervical level of the spine.
31. A system for treating chronic pain in a subject, the system comprising:
   a first electrode adapted for positioning at a location in the epidural space to deliver electrical stimulation to the spinal cord of a subject;
   a second electrode adapted for positioning at a frontal lobe region of the brain of the subject and for detecting a brain electrical signal from the frontal lobe region of the brain of the subject; and
   a processor programmed to instruct the first electrode to apply an electrical stimulation to the spinal cord in a manner effective to treat the chronic pain in the subject when a brain electrical signal that exceeds a threshold level is detected using the second electrode.
32. The system of aspect 31, wherein the frontal lobe region is a right frontal lobe region of the brain.
33. The system of aspect 31 or 32, wherein the brain electrical signal comprises alpha frequency, beta frequency, gamma frequency, delta frequency, or theta frequency neural oscillations.
34. The system of aspect 33, wherein the theta frequency neural oscillations are in a range from 4 Hz to 8 Hz.
35. The system of aspect 33, wherein the beta frequency neural oscillations are in a range from 12 Hz to 30 Hz.
36. The system of any one of aspects 31-35, wherein the second electrode is a non-brain penetrating surface electrode array or a brain-penetrating electrode array.
37. The system of any one of aspects 31-36, further comprising a third electrode adapted for positioning at a location in a left frontal cortex region of the brain of the subject.
38. The system of aspect 37, wherein the third electrode is a non-brain penetrating surface electrode array or a brain-penetrating electrode array.
39. The system of any one of aspects 31-38, wherein the system further comprises a user interface comprising an input electronically coupled to the processor for instructing the first electrode to apply an electrical stimulation to the spinal cord to treat the chronic pain in the subject.
40. The system of aspect 39, wherein the user interface is password protected and is operable by a health care practitioner.
41. The system of any one of aspects 31-40, wherein the chronic pain is caused by a pain-associated disorder, wherein applying the electrical stimulation relieves the pain.
42. The system of aspect 41, wherein the pain-associated disorder is back pain, failed back surgery syndrome, spinal cord injury, spinal stenosis, post-surgical pain, complex regional pain syndrome, arachnoiditis, angina, nerve-related pain, peripheral vascular disease, pain after an amputation, visceral abdominal pain, perineal pain, multiple sclerosis, arthritis, or chronic leg or arm pain.
43. The system of any one of aspects 31-42, wherein the processor is further programmed to modulate one or more programmed stimulation parameters according to the algorithm's control law; and apply the modulated electrical stimulation to the spinal cord using the first electrode in a manner effective to treat the chronic pain.
44. The system of any one of aspects 31-43, wherein the processor is further programmed to set a maximum number of electrical stimulations per day.
45. The system of any one of aspects 31-44, wherein the processor is further programmed to set a total amount of time of electrical stimulation per day.
46. A computer implemented method for programming a spinal cord stimulator (SCS) to relieve chronic pain in a subject, the computer performing steps comprising:
   a) receiving recorded brain electrical signal data from a frontal lobe region of the brain of the subject;
   b) analyzing the recorded brain electrical signal data using a pain classification model that identifies patterns of electrical signals in the recorded brain electrical signal data associated with the chronic pain;
   c) adjusting one or more programmed stimulation parameters based on the recorded brain electrical signal data according to an algorithm control law; and
   d) instructing the spinal cord stimulator to apply an electrical stimulation to the spinal cord to treat the chronic pain in the subject.
47. The computer implemented method of aspect 46, wherein a machine learning algorithm is used to generate the pain classification model.
48. The computer implemented method of aspect 47, wherein the machine learning algorithm is a support vector machine (SVM) learning algorithm.
49. The computer implemented method of any one of aspects 46-48, further comprising receiving recorded brain electrical signal data from a left frontal cortex region of a brain of the subject; and adjusting of one or more programmed stimulation parameters to maintain a level of a brain electrical signal associated with relief of the chronic pain in a target range.
50. The computer implemented method of any one of aspects 46-49, further comprising determining a minimum stimulation amplitude needed to relieve the chronic pain based on the recorded brain electrical signal data; and instructing the spinal cord stimulator to apply an electrical stimulation to the spinal cord at the minimum stimulation amplitude needed to relieve the chronic pain.
51. The computer implemented method of any one of aspects 46-50, further comprising receiving inputted data comprising a paresthesia threshold for the electrical stimulation; and instructing the spinal cord stimulator to apply the electrical stimulation at a level below the paresthesia threshold.
52. The computer implemented method of any one of aspects 46-51, further comprising processing the recorded brain electrical signal data to remove non-physiological frequencies from 1 Hz to 150 Hz.
53. The computer implemented method of any one of aspects 46-52, further comprising performing baseline correction on the recorded brain electrical signal data.
54. The computer implemented method of any one of aspects 46-53, further comprising removing ambient electrical line noise from the recorded brain electrical signal data.
55. The computer implemented method of aspect 54, wherein said removing ambient electrical line noise comprises using a Notch filter at 60 Hz and harmonics to remove the ambient electrical line noise.
56. The computer implemented method of any one of aspects 46-55, further comprising removing a first minute and a last minute from the recorded brain electrical signal data.
57. The computer implemented method of any one of aspects 46-56, further comprising performing independent component and time-locked coherence analyses to detect and reduce artifacts caused by blink and pulse activity.
58. The computer implemented method of any one of aspects 46-57, further comprising splitting the recorded brain electrical signal data into consecutive time epochs.
59. The computer implemented method of aspect 58, wherein each time epoch comprises 1 second of time of the recorded brain electrical signal data.
60. The computer implemented method of aspect 58 or 59, further comprising performing frequency decomposition to calculate average power in each neurophysiological activity frequency band for each time epoch.
61. The computer implemented method of any one of aspects 58-60, further comprising:
   calculating spectral tilt values for each epoch; and
   fitting the spectral tilt values to the pain classification model to determine how to adjust said one or more programmed stimulation parameters.
62. The computer implemented method of any one of aspects 58-61, further comprising:
   a) ranking predicted stimulation effectiveness for available settings of the spinal cord stimulator based on classifier scores for stimulation effectiveness of each setting using a linear classification model;
   b) selecting stimulation settings predicted to have highest stimulation effectiveness based on the linear classification model;
   c) receiving recorded brain electrical signal data from the frontal lobe region of the brain of the subject after applying electrical stimulation with the spinal cord stimulator to the spinal cord using the settings predicted to have highest stimulation effectiveness;
   d) analyzing the recorded brain electrical signal data to evaluate neural response of the subject to the electrical stimulation;
   e) updating the linear classification model based on the neural response of the subject to the electrical stimulation to generate an updated linear classification model;
   f) updating the ranking of predicted stimulation effectiveness for the available settings of the spinal cord stimulator using the updated linear classification model;
   g) selecting stimulation settings predicted to have the highest stimulation effectiveness based on the updated linear classification model;
   h) receiving recorded brain electrical signal data from the frontal lobe region of the brain of the subject after applying the electrical stimulation with the spinal cord stimulator to the spinal cord using the settings predicted to have highest stimulation effectiveness based on the updated linear classification model; and
   i) repeating e)-h) to adjust the available settings of the spinal cord stimulator to optimize stimulation effectiveness.
63. The computer implemented method of aspect 62, further comprising predicting whether a subject who has chronic pain will respond to the spinal cord stimulation based on the classifier scores for stimulation effectiveness.
64. The computer implemented method of any one of aspects 46-63, further comprising storing a user profile for the subject comprising information regarding the recorded brain electrical signal data associated with the chronic pain.
65. The computer implemented method of any one of aspects 46-64, further comprising storing a user profile for the subject comprising information regarding the programmed stimulation parameters used to apply electrical stimulation to the spinal cord to treat the chronic pain in the subject based on the recorded brain electrical signal data.
66. A non-transitory computer-readable medium comprising program instructions that, when executed by a processor in a computer, causes the processor to perform the method of any one of aspects 46-65.
67. A kit comprising the non-transitory computer-readable medium of aspect 66 and instructions for treating chronic pain in a subject with a spinal cord stimulator.
68. A system for treating chronic pain in a subject, the system comprising:
   a spinal cord stimulator comprising a first electrode adapted for positioning at a location in the epidural space to deliver electrical stimulation to the spinal cord of a subject;
   a neural recording device comprising a second electrode adapted for positioning at a right frontal lobe region of the brain of the subject for recording brain electrical signal data from the right frontal lobe region of the brain of the subject; and
   a processor programmed according to the computer implemented method of any one of aspects 46-65 to adjust one or more stimulation parameters based on the recorded brain electrical signal data and instruct the first electrode to apply an electrical stimulation to the spinal cord.
69. A kit comprising the system of aspect 68 and instructions for treating chronic pain in a subject with a spinal cord stimulator.
70. A method of detecting whether a subject who has chronic pain is responding to spinal cord stimulation therapy, the method comprising:
   positioning a first electrode at a location in the epidural space to deliver electrical stimulation to the spinal cord of the subject;
   positioning a second electrode at a location in a frontal lobe region of the brain of the subject to detect a brain electrical signal associated with the chronic pain;
   detecting the brain electrical signal in the frontal lobe region of the brain of the subject using the second electrode before and after applying electrical stimulation to the spinal cord using the first electrode, wherein a decrease in level of power of the brain electrical signal indicates the subject is responding to the spinal cord stimulation therapy and an increase or no change in the level of power of the brain electrical signal indicates the subject is not responding to the spinal cord stimulation therapy.

### EXAMPLES

As can be appreciated from the disclosure provided above, the present disclosure has a wide variety of applications. Accordingly, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, dimensions, etc.) but some experimental errors and deviations should be accounted for. Those of skill in the art will readily recognize a variety of noncritical parameters that could be changed or modified to yield essentially similar results.

### Example 1: An Automated Process for Optimizing Spinal Cord Stimulation Programming and Predicting SCS Trial Success

Chronic pain will affect 1 in 4 Americans in their lifetime (CDC (2016) Wide-ranging online data for epidemiologic research (WONDER)). Few studies exist evaluating the effect of spinal cord stimulation (SCS) on neural activity as measured with electroencephalography (EEG) in humans. EEG allows us to study endogenous neural patterns during various pain states and how they are affected by spinal cord stimulation. There is a fundamental gap in our knowledge of the central nervous system-level mechanism of action of SCS and how varying amplitudes may modify pain-related brain activity.

Of the patients that receive permanent SCS implants, over 70% report a reduction in pain of greater than 50%. However, SCS therapy trials following the current standard of care have an industry-wide failure rate as high as 52%.⁷ Further, of the patients that receive permanent SCS implants, most experience a reduction in efficacy greater than 50% within 2 years^{8,9}, potentially due to nervous system adaptation to electrical stimulation over time. Using EEG-guided programming sessions during initial programming would enable clinicians to efficiently and objectively determine a patient's ideal stimulation settings, thereby reducing the duration and increasing the success rate of the trial process. Additionally, finding the stimulation amplitude that provides pain relief without paresthesia would reduce the overall electrical dosage delivered during therapy. This reduction in electrical exposure would delay loss of efficacy due to nervous system adaptation, as well as extend the battery life of the implanted pulse generator to improve the time between recharges and/or replacements.

We have developed brain-based biomarkers of high pain states in individual patients using spinal cord stimulation (SCS) therapy to manage chronic pain. These personalized biomarkers are obtained using scalp recording with electroencephalography (EEG) and machine-learning tools that identify ‘neural signatures’ of chronic pain in individual patients. Neural signatures consist of patient-specific mathematical models that incorporate power of brain oscillations (e.g., theta (4-8 Hz) and beta band (12-30 Hz) signals) and spatial coherence measures (i.e., for network analysis). With concurrent EEG measurements taken during SCS programming sessions, we have identified patterns of brain activity in response to pain relief, which lends insight into how different stimulation patterns affect key nodes in pain-related brain circuits. These biomarkers successfully distinguish optimal SCS parameters from ineffective stimulation. Using expertise from neural engineering and signal analysis, we have developed a computational pipeline that allows automated filtering and preprocessing of EEG signals to produce these candidate neural signatures of pain state and SCS response in individual patients. Further, this method can be used to estimate optimal SCS programming parameters within individuals. By delivering optimal stimulation that is below the paresthesia threshold but still analgesic, electrical dosage may be reduced resulting in prolonged therapeutic effects of SCS and longer battery life. Finally, identifying which stimulation parameters may continue to provide pain relief even at lower amplitudes, we may tailor therapy to an individual's pain relief.

### Building the Baseline Model:

### Collect EEG Data

Subjects 18-99 years old with a chronic pain condition (CRPS, neuropathy, failed back surgery, etc.) successfully managed by a spinal cord stimulator (>50% pain relief) were recruited. The subjects are connected to high-frequency EEG measurement hardware (currently 64-channel headcap with exogenous ECG trace, 4 kHz sampling rate amplifier). Raw EEG data is recorded in segments of 5 minutes or longer with varying stimulation settings, including baseline (stimulation off), paresthesia threshold, optimal therapeutic stimulation, modified and ineffective stimulation, and maximum tolerated stimulation.

### Clean Raw EEG Data

Raw data is loaded into the software pipeline implementing functions from the open-source EEGLAB and Fieldtrip analysis packages and custom scripts deployed in MATLAB. Raw data is processed by performing the following steps in software:
1. Baseline correction to remove DC offset and linear trends.
2. Band pass filter between 1 Hz and 150 Hz to remove non-physiological frequencies from the data.
3. Notch filter at 60 Hz and harmonics to remove ambient electrical line noise, if needed.
4. Remove first and last minute from the data to isolate clean segments of data, then split into 1-second consecutive epochs Remove bad or defective channels from the dataset as needed by interpolating data from the surrounding usable channels.
5. Detect and reduce artifacts caused by blink and pulse activity using independent component and time-locked coherence analyses.
6. Re-reference data to common average reference or common median reference.

### Build Key EEG Feature Sets

Frequency decomposition is performed on the data to calculate the power spectrum for each contact and each epoch across all study trials. Full frequency decompositions are used to calculate average power in each neurophysiological activity band (delta, theta, alpha, beta, gamma etc.). The slope of 1/f power values is determined across frequency bands by fitting a curve to the band power data for each epoch (slope is calculated on 5-7 samples for each EEG contact and each time epoch, over the axes ‘frequency band’ vs ‘power’); this value has been called the ‘spectral tilt’ measure in previous literature. The above slope is used to reference standard values as established through our review. Training and testing data tables are created with spectral tilt values for each epoch as features and the associated stimulator setting types as labels.

### Build Classification Model

The training data is organized according to the desired validation scheme, with the full model using leave-one-out cross-validation. Autocorrelation analysis is performed to evaluate the impact of temporal correlations and determine the appropriate window of training data to omit (n=5) to avoid model overfitting. Maximum autocorrelation time lag determines time window of the data to omit. The training data is fit to a multiclass quadratic support vector machine (SVM) model and evaluated to assess accuracy.

### Deploying the Classification Model:

This section explores an initial set of potential uses for the baseline model defined using the methods above. Applications of this model include: i) recording EEG data from patients in the clinic or remotely using appropriate wired or wireless EEG hardware; ii) generating test data from the EEG recordings using some implementation of the software processing methods described above; and iii) applying the baseline classification model to the test data to evaluate the patient's condition or therapy.

The classification model and methods described above can be used for optimal parameter search (analgesia without paresthesia, minimum stimulation amplitude) by cycling through different stimulation settings while recording EEG data to find the subjective indicators that confirm effective therapy. In addition, appropriate and inappropriate candidates for stimulation therapy can be evaluated based on the output of the predicted probability of SCS trial success from SVM classifier trained on our validation datasets.

Collection of de-identified EEG data may be used for further analysis to identify biomarkers of pain conditions, pain states versus effective stimulation parameters, impact of different setting changes etc., and to update and improve the baseline classification model.

### Example 2: SCS Programming Session Using EEG Data for a Subject

Each subject undergoes one visit lasting 2 hours for EEG recording. The subject is asked to turn off their SCS device for a period of at least 8 hours prior to the session and answer standard questionnaires about pain and analgesia, including an assessment using the Visual Analog Score, Neuropathic and Nociceptive Pain Scale (NIH PROMIS). A 64-channel EEG headset is fitted to their head. EEG data is sampled at 2048 Hz using a 64+8-channel BioSemi ActiveTwo System (BioSemi Instrumentation, Netherlands). 7 conditions are tested in random order for 5 minutes each having the subject with eyes open fixating on a cross in the center of a black screen:

### 1. Baseline

### 2-7. Various Stimulation Amplitudes Above and Below a Pre-Determined Paresthesia Detection Threshold are Tested.

Personalized biomarkers are identified for the subject, obtained using the scalp recording with EEG and machine-learning tools that identify ‘neural signatures’ of chronic pain in individual patients. Neural signatures consist of patient-specific mathematical models that incorporate the power of brain oscillations (e.g. theta (4-8 Hz) and beta (12-30 Hz) band signals) and spatial coherence measures (i.e. for network analysis). With simultaneous EEG and SCS measurements during programming sessions, patterns of brain activity in response to pain relief are identified for the subject.

### Example 3: High-Frequency EEG Data for 8 Patients with Chronic Pain Conditions Managed by Spinal Cord Stimulation

To date, we have recruited and recorded high-frequency EEG data in 8 patients with chronic pain conditions managed by spinal cord stimulators. We are currently actively recruiting patients to meet our enrollment target, collecting and analyzing data, and further refining the biomarker and classification pipeline models.

Our current data processing and analysis pipeline includes activities to clean filter, and perform various transformations to produce training datasets for classification. First, high- and low-pass filters are applied to eliminate linear effects and non-physiological high-frequency electrical activity respectively. A notch filter at 60 Hz is also applied to remove ambient electrical line noise. The data is then cleaned by applying a constant baseline offset correction; removing extremely noisy or dead channels and interpolating, if necessary; removing any bad blocks or large segments of noise, signal disruption, and large movement artifacts obscuring all channels; and identifying and reducing blink and pulse artifacts using independent component analysis (ICA). These steps are currently performed using the open-source FieldTrip scripting toolbox. After this preprocessing, the data is exported for further analysis. The data is then loaded into EEGLAB—an open-source EEG analysis package implemented in MATLAB—for additional processing and analysis. First, the first and last minutes are removed and a segment of approximately 120 seconds of clean signal is isolated, then the remaining data is split into epochs of 1 second duration. The data is also re-referenced to a common average and updated with the relevant electrode contact location and label information. After this additional processing, we perform various transformations to produce datasets for further analysis. For example, we evaluate the spectral power density of each epoch, averaged within the physiological neural activity bands (delta, theta, alpha, beta, gamma). In another analysis, we fit a linear curve to the spectral power of each band, defining the spectral tilt value of each epoch as the slope of this curve. Next, using these calculations for each contact location as individual features and the set of values for each epoch as individual observations, we label each observation based on the corresponding spinal cord stimulator setting. We then apply a multi-class quadratic support vector machine (SVM) classification technique to train an algorithm to identify each trial and stimulator setting condition with far better than random accuracy. To improve the generalizability of the model and protect against overfitting the model to the training data, several techniques are employed to optimize the classification algorithm, including training data windowing and cross validation.

### REFERENCES

1. CDC. Wide-ranging online data for epidemiologic research (WONDER). 2016, (2016).
2. Shmagel, A., Foley, R. & Ibrahim, H. Epidemiology of chronic low back pain in US adults: National Health and Nutrition Examination Survey 2009-2010. Arthritis Care Res. 68, 1688-1694 (2016).
3. Kumar, K. et al. Spinal cord stimulation versus conventional medical management for neuropathic pain: a multicentre randomised controlled trial in patients with failed back surgery syndrome. Pain 132, 179-188 (2007).
4. Melzack, R. & Wall, P. D. Pain Mechanisms: A New Theory. Science 150, 971-979 (1965).
5. Bordeleau, M., Cottin, S. C., Meier, K. & Prud' Homme, M. Effects of Tonic Spinal Cord Stimulation on Sensory Perception in Chronic Pain Patients: A Systematic Review. Neuromodulation Technol. Neural Interface 0.
6. Koyama, S., Xia, J., Leblanc, B. W., Gu, J. W. & Saab, C. Y. Sub-paresthesia spinal cord stimulation reverses thermal hyperalgesia and modulates low frequency EEG in a rat model of neuropathic pain. Sci. Rep. 8, 7181 (2018).
7. Yang, F. et al. Modulation of Spinal Nociceptive Transmission by Sub-Sensory Threshold Spinal Cord Stimulation in Rats After Nerve Injury. Neuromodulation Technol. Neural Interface 0,
8. Wolter, T., Kiemen, A., Porzelius, C. & Kaube, H. Effects of sub-perception threshold spinal cord stimulation in neuropathic pain: A randomized controlled double-blind crossover study. Eur. J. Pain 16, 648-655 (2012).
9. Mekhail, N. et al. Paresthesia-Free Dorsal Root Ganglion Stimulation: An ACCURATE Study Sub-Analysis. Neuromodulation Technol. Neural Interface 0.

### Example 4: PATIENT USE CASE EXAMPLES

Indication/Symptoms: Chronic Low back pain with or without radiating leg pain
Possible ICD 10 Diagnoses (or Related to):
Spondylolysis (acquired): M43.06
Degenerative disc disease—Lumbar: M51.36 L/S: M51.37
Disc herniation—Lumbar: M51.26 or 27
Disc herniation with myelopathy—Lumbar: M51.06
Facet syndrome—Lumbar: M54.06 or M54.07
Failed Low back pain (Lumbago): M54.x
Radiculopathy—Lumbar: M54.16 L/S: M54.17
Radiculopathy 2/2 disc herniation—Lumbar or sacral M51.x
Spinal stenosis (Central): Lumbar central stenosis M48.x
Lumbar central stenosis due to disc herniation: M99.53
Spinal stenosis (Foraminal)—Lumbar—M99.x
Spondylolisthesis—Lumbar: M43.16
Spondylolysis (and congenital spondylolisthesis): Q76.2
back syndrome (post-laminectomy)—Lumbar: M96.1
Iliolumbar syndrome (sprain of lumbar ligaments): S33.5xxA, S33.5xxD

Clinical and/or Research Methods: The patient prepares for either Spinal cord stimulator invasive trial or permanent implant with any manufacturer.
1. Patient comes to facility or at home, uses a non-invasive EEG recording at resting state, while sitting quietly or going about activities for up to 30 minutes, with data from all contacts recorded by a computer.
2. The data is automatically cleaned and preprocessed as below.
3. The computer processes the EEG recording from the subject and then outputs a host of recommended stimulation parameters to the clinician or technician, including 1) contact location and configuration, 2) stimulation frequency 3) stimulation pulse width 4) stimulation amplitude
4. If needed, the patient will don the cap after insertion of electrodes and programmed settings will be manually programmed by the technician/clinician.
5. The computer will output updated optimal stimulation parameter estimates based on EEG recordings obtained during manual programming.
6. The EEG cap or computer may interface with the patient's handheld device or implantable stimulator to automatically update stimulation settings
7. Patient removes cap and is discharged from the office or session.

Indication/Symptoms: Pain in the legs or feet due to neuropathy, tingling, paresthesias or burning/numbness in the feet. Aching or dull pain in joints of the leg, Difficulty walking

### Possible ICD 10 Diagnoses (or Related to):

G90.52—Complex regional pain syndrome I of lower limb
E13.4x—Other specified diabetes mellitus with neurological complications including neuropathies
E11.x—Type 2 diabetes mellitus with diabetic polyneuropathy or any neuropathy
G90.0x—Idiopathic peripheral autonomic neuropathy
G60.x—Hereditary and idiopathic neuropathy
G61.x—Inflammatory polyneuropathy
G62.x—Other and unspecified polyneuropathies
G63.x—Polyneuropahy in diseases classified elsewhere
G64.x—Other disorders of peripheral nervous system
G65.x—Sequelae of inflammatory and toxic polyneuropathies

Clinical and/or Research Methods: The patient prepares for either Spinal cord stimulator invasive trial or permanent implant with any manufacturer.
1. Patient comes to facility or at home, uses a non-invasive EEG recording at resting state, while sitting quietly or going about activities for up to 30 minutes, with data from all contacts recorded by a computer.
2. The data is automatically cleaned and preprocessed as below.
3. The computer processes the EEG recording from the subject and then outputs a host of recommended stimulation parameters to the clinician or technician, including 1) contact location and configuration, 2) stimulation frequency 3) stimulation pulse width 4) stimulation amplitude
4. If needed, the patient will don the cap after insertion of electrodes and programmed settings will be manually programmed by the technician/clinician.
5. The computer will output updated optimal stimulation parameter estimates based on EEG recordings obtained during manual programming.
6. The EEG cap or computer may interface with the patient's handheld device or implantable stimulator to automatically update stimulation settings
7. Patient removes cap and is discharged from the office or session.
   Indication/Symptoms: Neck Pain with or without Radiation to the Head or Arms

### Possible Diagnoses (or Related to):

Cervicalgia (neck pain): M54.2
Degenerative disc disease (cervical): M50.3x
Disc herniation (cervical): M50.2x
Dystonia/Torticollis: M43.6
Facet syndrome (cervical): M54.02
Failed back syndrome (post-laminectomy)-Cervical: M96.1
Cervical or thoracic radiculopathy: M54.1x
Radiculopathy due to disc herniation (cervical): M50.1x
Spinal stenosis (cervical): M48.02
Spondylosis with or w/o radiculopathy or myelopathy: M47.x
Spondylolisthesis—Cervical: M43.1x

Clinical and/or Research Methods: The patient prepares for either Spinal cord stimulator invasive trial or permanent implant with any manufacturer.
1. Patient comes to facility or at home, uses a non-invasive EEG recording at resting state, while sitting quietly or going about activities for up to 30 minutes, with data from all contacts recorded by a computer.
2. The data is automatically cleaned and preprocessed as below.
3. The computer processes the EEG recording from the subject and then outputs a host of recommended stimulation parameters to the clinician or technician, including 1) contact location and configuration, 2) stimulation frequency 3) stimulation pulse width 4) stimulation amplitude
4. If needed, the patient will don the cap after insertion of electrodes and programmed settings will be manually programmed by the technician/clinician.
5. The computer will output updated optimal stimulation parameter estimates based on EEG recordings obtained during manual programming.
6. The EEG cap or computer may interface with the patient's handheld device or implantable stimulator to automatically update stimulation settings
7. Patient removes cap and is discharged from the office or session.

Indication/Symptoms: Any of the above, when the patient is being considered for a SCS implant, but is pending workup and evaluation to determine candidacy.

Clinical and/or Research Methods:

The patient is being evaluated for a Spinal cord stimulator—the following workflow will be used to guide clinical decision-making regarding probability of patients' therapeutic response to SCS.
1. Patient comes to facility or at home, uses a non-invasive EEG recording at resting state, while sitting quietly or going about activities for up to 30 minutes, with data from all contacts recorded by a computer.
2. The data is automatically cleaned and preprocessed as below.
3. The computer processes the EEG recording from the subject and then outputs statistics related to their pain phenotype and probability of future therapeutic success to Spinal cord stimulation therapy. The computer algorithm will compare key EEG features to the database and make a prediction. Success is defined as a reduction of overall pain by 50% or more or a clinically significant improvement in function in daily activities.

### Example 5: Building the Baseline Classification Model

After subjects arrived at the study facility and all necessary study enrollment activities were completed, data collection began. First, baseline surveys were administered to collect demographic data and assess chronic pain condition, severity, and impact on function. These surveys included the Visual Analog Scale (VAS), Numerical Rating Scale (NRS), Patient-Reported Outcomes Measurement Information System (NIH-PROMIS), and Beck's Depression Inventory (BDI). While completing these surveys, subjects were connected to the EEG collection hardware. For the purposes of raw data collection within the clinical study to build the baseline model, hardware included: the BioSemi ActiveTwo EEG Amplifier, a 64-channel, 10-20 configuration EEG headcap with associated pin-type active (Sn) electrodes; flat-type electrodes for pulse and stimulation artifact capture; a laptop running the BioSemi data acquisition software; and a video camera for motion artifact capture. Patients' heads were measured to select and fit the appropriate EEG headcap, and signal, reference, and exogenous electrode contacts were applied to the 64-channel headcap locations, CMS/DRL reference electrode locations, stimulation contact locations, and heart using conductive electrode gel. The amplifier was then connected to the BioSemi acquisition software and electrode offsets were evaluated to confirm appropriate conditions for data capture.

Before data capture began, the desired stimulation conditions and settings were identified. These conditions included the subjects' typical therapeutic stimulation settings, typical therapeutic settings with reduced stimulation amplitude, ineffective stimulation settings, and high-amplitude stimulation settings. These stimulation conditions were organized according to a pre-determined, randomized order and raw EEG data was recorded in intervals of 5-minutes for each condition, beginning with the device stimulation turned off. In addition to the EEG data, the following information was recorded for each interval: individual stimulation setting values, including contact and location, frequency (Hz), pulse width (us), and amplitude (mA); subject pain response; and ECG and stimulation waveforms. Data was streamed from the amplifier to the data acquisition software at a rate of 4 KHz, and intervals were recorded as separate files and saved to encrypted, backed-up storage media for processing.

For initial cleaning and processing of the raw datasets, an automated computational pipeline was developed and deployed using open-source EEG analysis packages implemented in the MATLAB computing environment. Raw datasets were loaded into the pipeline and initially cleaned to produce adequate data for analysis. Baseline correction was first performed to remove DC offset effects and linear trends from the data. The data was then band-pass filtered between 1 Hz and 150 Hz to remove to remove non-physiological frequencies and notch filtered at 60 Hz to remove electrical line noise from the ambient environment. Bad or defective electrode channels were removed from the datasets and replaced by interpolating the surrounding usable channels. The first and last minute of the recording were removed to isolate the typically most stable region of the data, then split into consecutive epochs of 1 second in duration. Artifacts generated by disturbances during the recording, such as pulses from the subject's heart or blinks, eye and jaw motion, were identified using thresholding and Z-score analysis on select channels. Once these artifacts were identified, independent component analysis (ICA) was performed on the data to isolate individual statistically independent components, followed by time-locked coherence analyses to highlight the components generated by the artifact signal. These artifactual components were then removed to reduce the artifact signal from the original data stream. Finally, the dataset was re-referenced to the common average and labeled according to its effective stimulation condition.

Frequency decomposition was performed on the cleaned datasets to calculate the per-channel power spectrum for each epoch across all study trials. These frequency decompositions were then used to calculate the average power in each neurophysiological activity band (delta, theta, alpha, beta, gamma etc.). The slope of 1/f power values across frequency bands was determined for a given epoch by fitting a curve to the band power data for that epoch (slope is calculated on 5-7 samples for each EEG contact and each time epoch, over the axes ‘frequency band’ vs ‘power’); this value has been called the ‘spectral tilt’ measure in previous literature. This slope is the metric used to reference standard values as established through prior review. After cleaning and processing the available raw datasets, data tables are created with the spectral tilt values for each epoch as features and the associated stimulation conditions as labels.

These data tables were then prepared and processed using a machine learning analysis pipeline implemented in the MATLAB computing environment. An autocorrelation analysis was performed on the sequenced data to evaluate the impact of temporal correlations and determine an appropriate window of training data to omit from the validated models to avoid overfitting. Once the training and test data tables were arranged using the windowing above, the training datasets were fit to a multi-class quadratic support vector machine (SVM) model and cross-validated to assess model performance. The feature weight matrix produced by this SVM model is the element which can then be used to generate the predictive scores that determine data classifications.

Neural features from EEG data collected with the subjects' stimulation devices turned off were also isolated and processed with a subspace identification method. This analysis defines the characteristics of a subject's baseline, high-pain condition as the latent state features of a linear state space model. Subspace identification was also performed on labeled neural features from EEG data collected during spinal cord stimulation to characterize the input-driven model dynamics. Defining these intrinsic and input-driven dynamics allowed the creation of a predictive linear model that estimates neural feature response based on the current neural state and input spinal cord stimulation parameters. This model facilitates the direct prediction of optimal spinal cord stimulation settings.

### Example 6: Spinal Cord Stimulation Parameter Programming/Adjustment Optimization Session

The neural biomarker and associated classification and predictive model described here can be utilized to guide spinal cord stimulator (SCS) initial programming or reprogramming sessions to optimize stimulation parameters. By collecting EEG data from chronic pain patients with SCS devices and applying the model described above, clinicians can quickly identify the stimulation parameters likely to produce therapeutic pain relief. This approach would highlight the most potentially effective groups of stimulation parameters without relying solely on the patient's subjective feedback of pain perception and response, and without multiple rounds of trial-and-error adjustments by the clinicians or field engineers.

Data collection for this process varies according to the setting of the programming session. In a clinical setting, such as a hospital, outpatient facility or pain management clinic, the collection equipment can match the hardware used in the clinical study to capture the necessary raw data. For a non-clinical setting, such as the patient's home or residential facility, the minimum equipment required for implementation would consist of any EEG hardware capable of recording data at a sampling rate of at least 512 Hz in segments of at least 30 seconds in duration. This could include wired or wireless amplifier systems, wet or dry surface electrodes with or without conductive gel, headcaps with standard 10-20 configuration or custom arrays with the subset of electrodes required to capture the relevant neural biomarkers, and systems with on-board raw data storage or with real-time data streaming capabilities. To implement the system as described, suitable EEG data collection equipment can include hardware with any combination of the features described above meeting the minimum data capture requirements stated. This equipment would be capable of producing segments of raw EEG data suitable for processing and classification.

Data processing for this implementation utilizes the same automated software pathway described in the baseline model. First, raw data is parsed and processed through a data cleaning pipeline that automatically performs a sequence of actions including baseline correction, artifact reduction, filtering and referencing. Clean data segments are then converted to unlabeled test datasets using the frequency decomposition pathway described in the base model to generate the features required for classification. Feature sets from data segments collected without stimulation are analyzed first to define individual, patient-specific latent state characteristics and update the dynamic model. Using this updated linear model, a forward prediction process is applied to the space of available stimulation configurations as inputs to produce an estimated neural response of the identified classification features. Feature weights from the classification model are applied to these feature sets to calculate their individual class scores and predict the corresponding stimulation condition label. The available stimulation configurations are ranked based on their classifier scores for optimal therapeutic stimulation, then individual groups of stimulation settings can be programmed into the patient's device for testing, feedback, and additional data collection and updates to the patient-specific model. This evaluation using the baseline classification and linear predictive models can be implemented on any computing platform capable of running compiled code, including any modem personal computing device such as a laptop, tablet, or mobile phone, or a cloud server or service. In the case where EEG data is streamed from the collection device in real-time, the automated data processing occurs concurrently as raw data is streamed, annotated, and stored to a companion storage system or remote service. When raw EEG data is stored to on-board device storage, data processing proceeds in a post-hoc fashion as raw data segments are loaded from a removable storage media onto an independent computing device for processing and analysis. The technical flexibility of this implementation allows a number of different embodiments across classes of equipment suitable for performing the raw neurological data collection and for supporting the software analysis and evaluation pipeline.

In one potential embodiment, a session of this biomarker-guided SCS programming procedure would involve:
Patient arrives at the healthcare facility for a device programming or parameter adjustment session.
Patient is connected to the EEG data collection hardware and raw data capture begins.
Raw data is streamed to companion processing device where automated software pipeline analyzes EEG neural features in the absence of stimulation to update linear model.
Software pipeline applies linear model to predict and rank stimulation effectiveness across space of available device settings.
Patient's SCS device is enabled at highest ranking stimulation settings Raw data is captured under these stimulation conditions for a duration of 30 seconds or more and streamed to the companion data processing device.
Stimulation condition for current setting is labeled according to the classification model response and patient feedback is confirmed.
Neural feature response to input stimulation is confirmed by newly captured EEG data, used to update model characteristics and predictive rankings.
Stimulation settings are adjusted as indicated by the classification model rankings.
EEG raw data capture and analysis is repeated for updated stimulation conditions until the biomarker for therapeutic stimulation is detected and patient feedback is confirmed.

The outcome metrics for the baseline classification model would match those calculated within the initial clinical study, including overall classifier accuracy, True-Positive-Ratio vs. False-Positive-Ratio, AUC, linear predictive error and correlation coefficient as evaluated by cross-validation across training datasets. In implementation, the performance of this system would be evaluated based on the individual predicted classifier scores, patient symptom reduction based on survey responses, and metrics associated with the functionality of the implanted SCS. These functionality metrics could include the total time required to reprogram the SCS device, the frequency of required device reprogramming sessions, and the time to loss of therapeutic benefit from stimulation. Furthermore, the session results and additional data recorded can be added to the baseline classification model to improve its accuracy or personalize it for that individual patient.

### Example 7: Spinal Cord Stimulator Trial Session

In another embodiment, the neural biomarker and classification model produced by analyzing spinal cord stimulator (SCS) trial data can be used to predict whether a chronic pain patient will respond to SCS therapy. By collecting baseline EEG data from a patient considering an SCS device for treatment of a chronic pain condition and applying the processing and classification model, the system can detect the appropriate biomarkers and determine the likely outcome of a trial procedure. The typical SCS trial is an undertaking that currently requires insurance and psychology approvals, equipment including a trial pulse generator and electrodes, and extensive follow-up and programming sessions with clinicians and device representatives. The approach suggested will predict whether a patient is likely to experience pain relief from SCS without going through this time-consuming, costly, and potentially fruitless process.

Data collection for this implementation would proceed using the same materials and process presented in the ‘SCS Parameter Optimization Session’ section above. Similarly, data cleaning and feature calculations are performed by the same automated software pipeline using the same computational platforms described in this previous implementation example. After datasets with the required features are created from the baseline, high-pain-state raw EEG data captured, feature weights from the trial prediction model are applied to determine the final classifier score. This classifier score is then assessed to determine the strength of the neural biomarker and predict the likelihood of SCS therapy success. Again, the technical flexibility of this implementation allows a number of different embodiments across the classes of suitable equipment available for data acquisition and analysis, as described in the previous implementation example.

In one potential embodiment of this implementation, a session of the biomarker-guided SCS trial prediction procedure would involve:
Patient arrives at healthcare facility for SCS trial evaluation.
Patient is connected to the EEG data collection hardware and raw data capture begins.
Raw data is captured under the patient's standard baseline pain-state conditions for a duration of 30 seconds or more and streamed to a companion device for automated data processing.
Patient neural activity under baseline chronic pain condition is processed and classified using the feature weights in the SCS Trial Prediction Model.
Classifier results are evaluated to predict the patient's likelihood of pain relief from SCS.

As in the implementation example above, outcome metrics for the trial prediction model would again match those calculated within the initial clinical study, including overall classifier accuracy as evaluated by cross-validation across training datasets. In implementation, the performance of this system would be evaluated based on the predicted classifier scores and metrics associated with the efficiency and accuracy of the biomarker-guided trial process. These metrics could include the total time required for the trial session and statistics and clinical outcomes related to the trial predictions. Furthermore, session results and additional data recorded can be added to the classification model over time to improve its accuracy for future patients.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method for treating chronic pain in a subject, the method comprising:
positioning a first electrode at a location in the epidural space to deliver electrical stimulation to the spinal cord of the subject;
positioning a second electrode at a location in a frontal lobe region of the brain of the subject to detect a brain electrical signal associated with the chronic pain;
detecting the brain electrical signal at the frontal lobe region of the brain of the subject using the second electrode; and
applying electrical stimulation to the spinal cord using the first electrode in a manner effective to treat the chronic pain in the subject when the brain electrical signal detected using the second electrode exceeds a threshold level.

2. The method of claim 1, further comprising using a control algorithm to automate said applying electrical stimulation when the brain electrical signal exceeds a threshold level.

3. The method of claim 2, wherein the control algorithm uses a machine learning algorithm for pain classification.

4-5. (canceled)

6. The method of claim 2, wherein the control algorithm further modulates one or more programmed stimulation parameters based on a level of power of the brain electrical signal, wherein the control algorithm further determines the minimum stimulation amplitude needed to relieve the chronic pain based on a level of power of the brain electrical signal.

7. (canceled)

8. The method of claim 1, wherein said applying the electrical stimulation comprises applying the electrical stimulation to the spinal cord at the minimum stimulation amplitude needed to relieve the chronic pain.

9. The method of claim 1, further comprising positioning a plurality of electrodes at the location in the frontal lobe region of the brain of the subject for detection of the brain electrical signal by stereoelectroencephalography (sEEG).

10. The method of claim 1, wherein the frontal lobe region is a right frontal lobe region of the brain.

11. (canceled)

12. The method of claim 1, wherein the brain electrical signal comprises alpha frequency, beta frequency, gamma frequency, delta frequency, or theta frequency neural oscillations.

13-14. (canceled)

15. The method of claim 1, wherein the second electrode is placed on a surface of a right frontal lobe region or within a right frontal lobe region, wherein the second electrode is a non-brain penetrating surface electrode array or a brain-penetrating electrode array.

16. (canceled)

17. The method of claim 1, further comprising positioning a third electrode at a location in a left frontal cortex region of the brain of the subject to detect a brain electrical signal associated with relief of the chronic pain.

18. The method of claim 17, wherein the third electrode is placed on a surface of the left frontal cortex region or within the left frontal cortex region, wherein the third electrode is a non-brain penetrating surface electrode array or a brain-penetrating electrode array.

19. (canceled)

20. The method of claim 1, further comprising using a control algorithm to automate adjustment of one or more programmed stimulation parameters to maintain the level of the brain electrical signal associated with relief of the chronic pain in a target range.

21. The method of claim 1, further comprising determining a paresthesia threshold for the electrical stimulation; and using a control algorithm to automate adjustment of one or more programmed stimulation parameters to apply the electrical stimulation at a level below the paresthesia threshold.

22. The method of claim 1, wherein the chronic pain is caused by a pain-associated disorder, wherein applying the electrical stimulation relieves the pain.

23-24. (canceled)

25. The method of claim 1, wherein
the method further comprises;
assessing effectiveness of the treatment in the subject; mapping the brain of the subject to identify an optimal location in the right frontal lobe region to detect the brain electrical signal associated with the chronic pain;
mapping the brain of the subject to identify an optimal location in the left frontal cortex region to detect the brain electrical signal associated with relief of the chronic pain;
assessing relief of pain during or after treatment of the subject by using a visual analog scale or a verbal rating scale; and/or
repositioning the first electrode in the epidural space to improve relief of pain.

26-30. (canceled)

31. A system for treating chronic pain in a subject, the system comprising:
a first electrode adapted for positioning at a location in the epidural space to deliver electrical stimulation to the spinal cord of a subject;
a second electrode adapted for positioning at a frontal lobe region of the brain of the subject and for detecting a brain electrical signal from the frontal lobe region of the brain of the subject; and
a processor programmed to instruct the first electrode to apply an electrical stimulation to the spinal cord in a manner effective to treat the chronic pain in the subject when a brain electrical signal that exceeds a threshold level is detected using the second electrode.

32. The system of claim 31, wherein the frontal lobe region is a right frontal lobe region of the brain.

33-35. (canceled)

36. The system of claim 31, wherein the second electrode is a non-brain penetrating surface electrode array or a brain-penetrating electrode array.

37. The system of claim 31, further comprising a third electrode adapted for positioning at a location in a left frontal cortex region of the brain of the subject, wherein the third electrode is a non-brain penetrating surface electrode array or a brain-penetrating electrode array.

38. (canceled)

39. The system of claim 31, wherein the system further comprises a user interface comprising an input electronically coupled to the processor for instructing the first electrode to apply an electrical stimulation to the spinal cord to treat the chronic pain in the subject.

40. (canceled)

41. The system of claim 31, wherein the chronic pain is caused by a pain-associated disorder, wherein applying the electrical stimulation relieves the pain.

42. (canceled)

43. The system of claim 31, wherein the processor is further programmed to modulate one or more programmed stimulation parameters according to the algorithm's control law; and apply the modulated electrical stimulation to the spinal cord using the first electrode in a manner effective to treat the chronic pain.

44. The system of claim 31, wherein the processor is further programmed to set a maximum number of electrical stimulations per day and/or a total amount of time of electrical stimulation per day.

45. (canceled)

46. A computer implemented method for programming a spinal cord stimulator (SCS) to relieve chronic pain in a subject, the computer performing steps comprising:
a) receiving recorded brain electrical signal data from a frontal lobe region of the brain of the subject;
b) analyzing the recorded brain electrical signal data using a pain classification model that identifies patterns of electrical signals in the recorded brain electrical signal data associated with the chronic pain;
c) adjusting one or more programmed stimulation parameters based on the recorded brain electrical signal data according to an algorithm control law; and
d) instructing the spinal cord stimulator to apply an electrical stimulation to the spinal cord to treat the chronic pain in the subject.

47-65. (canceled)

66. A non-transitory computer-readable medium comprising program instructions that, when executed by a processor in a computer, causes the processor to perform the method of claim 46.

67. A kit comprising the non-transitory computer-readable medium of claim 66 and instructions for treating chronic pain in a subject with a spinal cord stimulator.

68. A system for treating chronic pain in a subject, the system comprising:
a spinal cord stimulator comprising a first electrode adapted for positioning at a location in the epidural space to deliver electrical stimulation to the spinal cord of a subject;
a neural recording device comprising a second electrode adapted for positioning at a right frontal lobe region of the brain of the subject for recording brain electrical signal data from the right frontal lobe region of the brain of the subject; and
a processor programmed according to the computer implemented method of claim 46 to adjust one or more stimulation parameters based on the recorded brain electrical signal data and instruct the first electrode to apply an electrical stimulation to the spinal cord.

69. A kit comprising the system of claim 68 and instructions for treating chronic pain in a subject with a spinal cord stimulator.

70. A method of detecting whether a subject who has chronic pain is responding to spinal cord stimulation therapy, the method comprising:
positioning a first electrode at a location in the epidural space to deliver electrical stimulation to the spinal cord of the subject;
positioning a second electrode at a location in a frontal lobe region of the brain of the subject to detect a brain electrical signal associated with the chronic pain; and
detecting the brain electrical signal in the frontal lobe region of the brain of the subject using the second electrode before and after applying electrical stimulation to the spinal cord using the first electrode, wherein a decrease in level of power of the brain electrical signal indicates the subject is responding to the spinal cord stimulation therapy and an increase or no change in the level of power of the brain electrical signal indicates the subject is not responding to the spinal cord stimulation therapy.
